(19) United States
(12) Patent Application Publication
(10) Pub. No.: US 2016/0030234 A1
(43) Pub. Date: Feb. 4, 2016

(54) DEVICES, SYSTEMS AND METHODS OF COOLING THE SKIN

(71) Applicant: GENTHERM INCORPORATED, Northville, MI (US)

(72) Inventors: John Lofy, Claremont, CA (US); David Marquette, Farmington Hills, MI (US)

(21) Appl. No.: 14/775,039

(22) PCT Filed: Mar. 11, 2014
(86) PCT No.: PCT/US14/23703

§ 371 Date: Sep. 11, 2015

Related U.S. Application Data
(60) Provisional application No. 61/777,629, filed on Mar. 12, 2013.

(51) Int. Cl. A61F 7/00

(52) U.S. Cl. CPC A61F 7/007; A61F 2007/0055; A61F 2007/0095; A61F 2007/0091

(57) ABSTRACT

According to some embodiments, a thermal conditioning system for selectively cooling skin of a subject comprises at least one thermal conditioning device comprising a first side and second side, the second side being generally opposite of the first side. In some embodiments, the system further comprises a heat sink positioned along the second side of the at least one thermal conditioning device, wherein the first side of the at least one thermal conditioning device is configured to be placed in contact with or in close proximity to a skin surface of the subject to selectively cool or heat the skin surface.

### BACKGROUND

1. Field

This application relates to thermal conditioning devices, systems and methods, and more specifically, to devices, systems and methods for the therapeutic use of cooling and/or heating for treating a subject (e.g., human, other mammalian, etc.) and/or for other medical treatments.

2. Description of the Related Art

Cooling of the human or other mammalian body or skin can provide one or more benefits, particularly in situations or circumstances where a subject's skin and/or body temperature is elevated relative to normal, acceptable, comfortable and/or safe levels. For example, in some instances, a person's skin and/or body temperature may be undesirably high because of fever and/or another medical condition (e.g., infection, allergy or other adverse reaction, disease, etc.). In other cases, a subject's temperature may be elevated due to exposure to heat or sun and/or other source of heat (e.g., workspace). In other circumstances, it may be desirable to heat a person's skin or other anatomical location, either in lieu of or in addition to cooling, as desired or required. For example, heating can be used to treat hypothermia, chills and/or any other condition or ailment. Therefore, a need exists to provide devices, systems and methods of cooling and/or heating skin and/or other portions of a subject's anatomy.

### SUMMARY

According to some embodiments, a thermal conditioning system for selectively cooling and/or heating skin of a subject comprises at least one thermal conditioning device comprising a first side and second side, the second side being generally opposite of the first side. In some embodiments, the system further comprises a heat sink positioned along the second side of the at least one thermal conditioning device, wherein the first side of the at least one thermal conditioning device is configured to be placed in contact with or in close proximity to a skin surface of the subject to selectively cool or heat the skin surface. In some embodiments, the heat sink comprises one or more heat transfer members (e.g., fins, pins, etc.).

According to some embodiments, a method of selectively cooling and/or heating skin of a subject includes placing a thermal conditioning system adjacent a skin surface of a subject, wherein the thermal conditioning system comprises at least one thermal conditioning device (e.g., a thermoelectric device, a convective heater, a heat pump, another heating or cooling device, etc.) having a first side and second side, wherein the second side being generally opposite of the first side, wherein the thermal conditioning system further comprises a heat sink positioned along the second side of the at least one thermal conditioning device, wherein the first side of the at least one thermal conditioning device is configured to be placed in contact with or in close proximity to a skin surface of the subject to selectively cool or heat the skin surface.

According to some embodiments, the method further comprises activating the at least one thermal conditioning device so s to selectively heat or cool the subject's skin, and deactivating the at least one thermal conditioning device after a time period (e.g., 0-10 seconds, 10-20 seconds, 20-30 seconds, 30-45 seconds, 45-60 seconds, 1-2 minutes, 2-3 minutes, 3-5 minutes, 5-10 minutes, 10-15 minutes, 15-20 minutes, 20-30 minutes, 30 minutes-60 minutes, 1-1.5 hours, 1.5-2 hours, 2-3 hours, 3-4 hours, 4-5 hours, 5-10 hours, more than 10 hours, etc.). In some embodiments, the system is removably fastened to the subject using at least one fastener (e.g., strap, connector, buckles, clamp, clasp, etc.). In one embodiment, the system is positioned against or near the subject without fastening or otherwise securing the system to the subject.

According to some embodiments, the method further comprises detecting a temperature of the at least one thermal conditioning device and/or the subject's skin using at least one sensor (e.g., temperature sensor, thermocouple, etc.). In some embodiments, the system is configured to deactivate, at least in part (e.g., one or more of the thermal conditioning devices are deactivated), when a threshold temperature is detected by the at least one sensor. In some embodiments, the thermal conditioning system comprises a plurality of thermal conditioning devices, the thermal conditioning devices being arranged in at least two zones, wherein each of the at least two zones can be separately controlled and operated during use.

According to some embodiments, the at least one thermal conditioning device comprises a thermoelectric device (e.g., Peltier circuit or device). In some embodiments, the at least one thermal conditioning device comprises a convective heater, a heat pump and/or any other type of heating or cooling device. In some embodiments, the first side of the at least one thermal conditioning device is configured to contact, at least partially, a skin surface of the subject. In some embodiments, the first side of the at least one thermal conditioning device is configured to not contact the skin surface. Therefore, the system can be positioned, during use, so as to provide a desired clearance or spacing from the subject's skin.

According to some embodiments, the system further comprises at least one spacer or other feature, device or member configured to maintain a clearance between the first side of the at least one thermal conditioning device and the skin surface of the subject during use. In one embodiment, the spacer comprises a smooth surface and/or a slidable or other movable feature or portion. In some embodiments, such a clearance distance can be 1 mm to 20 mm (e.g., 1, 2, 3, 4, 5, 10, 15, 20 mm, values between the foregoing, etc.). In other embodiments, the clearance is less than about 1 mm (e.g., 0.01, 0.05, 0.1, 0.2, 0.3, 0.4, 0.5, 0.6, 0.7, 0.9, 0.9 mm, values between the foregoing, etc.) or more than about 20 mm (e.g., 21, 22, 25, 30, 35, 40, 50, 60 mm, more than 60 mm, values between the foregoing, etc.).

According to some embodiments, the system further comprises at least one intermediate member or base configured to support the at least one thermal conditioning device, wherein the at least one intermediate member or base is configured to at least partially contact the skin surface of the subject during use. In some embodiments, the at least one intermediate member or base comprises a layer of fabric, plastic or the like. In some embodiments, the base can be shaped, sized and otherwise configured to fit on or around a portion of the subject's body (e.g., particular surface, limb, etc.). In some embodiments, the at least one intermediate member or base is at least partially flexible so as to generally conform to a shape of the subject's skin surface.

According to some embodiments, the system comprises a plurality of (e.g., 2, 3, 4, 5, 6, 7, 8, 9, 10, 10-15, 15-20, more than 20, etc.) thermal conditioning devices. In one embodiment, at least two of the plurality of thermal conditioning devices are electrically and/or mechanically coupled to each other by at least one interconnecting member (e.g., post, wire, other rigid, semi-rigid or flexible member or feature, etc.). In some embodiments, the system further comprises at least one joint or movable feature (e.g., hinge, bendable portion or feature, etc.) along or near the at least one interconnecting member to provide additional flexibility to the system. In some embodiments, the thermal conditioning devices are arranged in a hexagonal pattern. In some embodiments, the thermal conditioning devices are arranged in a rectangular, triangular, other polygonal, circular (e.g., along one or more concentric circles), oval (e.g., along one or more concentric ovals), irregular, etc. pattern.

According to some embodiments, the system further comprises at least one sensor (e.g., temperature sensor, a humidity sensor, a condensation sensor, a pressure, contact or occupant sensor, etc.). In some embodiments, the system additionally includes at least one thermal switch or fuse configured to automatically cease power delivery to the at least one thermal conditioning device when a temperature associated with the at least one thermal switch or fuse rises above a maximum threshold or drops below a minimum threshold. In some embodiments, the system further includes thermal insulation (e.g., layers, components, etc.) along one or more outer surfaces and/or other portions of the heat sink (e.g., fins, pins, base, etc.).

According to some embodiments, the system further includes at least one of a power supply and a control module, wherein the power supply is configured to electrically activate the at least one thermal conditioning device, and wherein the control module is configured to regulate at least one aspect of the system. In one embodiment, the power supply comprises a battery, a connection to an AC or DC power supply and/or the like.

According to some embodiments, the at least one thermal conditioning device is configured to only cool the subject's skin. In some embodiments, the at least one thermal conditioning device is configured to only heat the subject's skin. In other embodiments, the at least one thermal conditioning device is configured to selectively cool and/or heat the subject's skin.

According to some embodiments, the system further comprises at least one fastener (e.g., strap, buckle, latch, etc.) configured to removably secure the system to the subject during use. In some embodiments, at least one component of the system comprises a non-planar or non-linear (e.g., curved, irregular, undulating, etc.) surface to enable the system to better conform to a shape of the subject's skin. In some embodiments, the at least one thermal conditioning device and the heat sink are collectively assembled into a single thermal conditioning assembly. In some embodiments, the plurality of thermal conditioning devices are divided or assembled into at least two zones (e.g., 2, 3, 4, 5, 6, 7, 9, 10, more than 10, etc.), each of the at least two zones being configured to be controlled independently of at least one other zone.

### BRIEF DESCRIPTION OF THE DRAWINGS

These and other features, aspects and advantages of the present application are described with reference to drawings of certain embodiments, which are intended to illustrate, but not to limit, the concepts disclosed herein. The attached drawings are provided for the purpose of illustrating concepts of at least some of the embodiments disclosed herein and may not be to scale.

FIG. 1 schematically illustrates a perspective view of one embodiment of a thermal conditioning system configured for use on a subject's limb;

FIG. 2 schematically illustrates a front perspective view of one embodiment of a thermal conditioning system configured for use on a subject's limb;

FIG. 3 schematically illustrates a side perspective view of one embodiment of a thermal conditioning system configured for use on a subject's limb;

FIG. 4 schematically illustrates a side view of one embodiment of a heat sink of a thermal conditioning system;

FIG. 5 illustrates a perspective view of a heat sink comprising one or more layers of thermal insulation according to one embodiment;

FIG. 6 schematically illustrates a perspective view of another embodiment of a thermal conditioning system configured for use on a subject's limb;

FIG. 7 schematically illustrates one embodiment of a heat sink of a thermal conditioning device comprising a plurality of pins;

FIG. 8a schematically illustrates a side view of one embodiment of a thermal conditioning device comprising a sensor;

FIG. 8b schematically illustrates a side view of one embodiment of a thermal conditioning device comprising at least one spacer to provide clearance between the device and the adjacent skin surface;

FIG. 8c schematically illustrates a side view of one embodiment of a thermal conditioning device comprising an intermediate or base layer positioned between the thermal conditioning device and the subject's skin;

FIG. 8d schematically illustrates a side view of one embodiment of a thermal conditioning device comprising a thermal insulation layer;

FIG. 9 schematically illustrates a perspective view of one embodiment of a glove-shaped thermal conditioning system configured to cool and/or heat a subject's hand;

FIG. 10 schematically illustrates a side view of thermal conditioning assemblies positioned along a subject's arm, according to one embodiment;

FIG. 11 schematically illustrates a thermal conditioning system comprises a plurality of thermal zones;

FIG. 12 schematically illustrates a side view of thermal conditioning assemblies according to one embodiment;

FIG. 13 schematically illustrates a side view of curved (e.g., non-linear) thermal conditioning assemblies according to one embodiment;

FIG. 14 schematically illustrates a top view of a thermal conditioning system comprises a plurality of thermal conditioning assemblies generally arranged in a hexagonal layout or pattern;

FIG. 15 schematically illustrates thermal conditioning assemblies forming a generally triangular pattern and having interconnecting members connecting each other according to one embodiment;

FIG. 16 schematically illustrates one embodiment of two interconnecting members attached to a thermal conditioning assembly;

FIG. 16b schematically illustrates one embodiment of a thermal conditioning system comprising a thermally conductive pad or mat;

FIG. 17 schematically illustrates one embodiment of a thermal conditioning system positioned around a limb of a subject and comprising at least one fastener to keep the system in place, relative to the subject, during use;

FIG. 18 schematically illustrates a perspective view of one embodiment of a thermal conditioning system configured for use on a subject's limb;

FIG. 19 schematically illustrates a front elevation view of the thermal conditioning system of FIG. 18;

FIG. 20 schematically illustrates an embodiment of a channel;

FIG. 21 schematically illustrates a perspective view of one embodiment of an expandable bladder for use with a thermal conditioning system; and

FIG. 22 schematically illustrates one embodiment of a control system for a thermal conditioning system.

### DETAILED DESCRIPTION

This application is generally directed to thermal conditioning systems for the cooling (and/or heating) of skin and/or other portions of a person's anatomy. Accordingly, the system and the various devices, systems and features associated with it are described herein in the context of human anatomy and/or therapeutic cooling or heating applications because they have particular utility in this context. However, the devices, systems and the methods described herein, as well as their various systems and features, can be used in other contexts as well, such as, for example, but without limitation, cooling or temperature regulation of surfaces and/or other portions of living or non-living beings or things, such as, for example, animals, plants, electronic devices or components, seating assemblies and/or the like.

Various embodiments disclosed herein utilize one or more thermoelectric devices to selectively cool and/or heat a part of the human anatomy, such as a skin surface or other tissue. In alternative embodiments, the thermal conditioning of skin or a target surface can be accomplished using one or more other thermal conditioning devices (e.g., cooling, heating and/or ventilation devices), such as, for example, heat pumps, convective heaters, refrigerant-based systems, other thermal conditioning device, etc., either in lieu of or in addition to thermoelectric devices. In some embodiments, a thermoelectric device (TED) comprises a Peltier device or circuit. According to some embodiments, a thermoelectric device comprises a first substrate and a second substrate disposed apart from each other. In some embodiments, the first and second substrates can be configured to provide electrical insulation (e.g., between adjacent layers or members). In addition, the thermoelectric device can comprise a plurality of semiconductor elements (e.g., pellets) comprising a first set of semiconductor elements and a second set of semiconductor elements, the first and second sets of semiconductor elements can include dissimilar electrical properties. In some embodiments, each of the semiconductor elements include a first end positioned toward the first substrate and a second end positioned toward the second substrate. In some embodiments, electrical conductors are used to electrically couple the ends of two adjacent semiconductor elements to one another. In some embodiments, the conductors are arranged and otherwise configured to electrically connect the semiconductor elements of the thermoelectric device to one another in series.

In some embodiments, when a voltage is applied to the thermoelectric device, the thermoelectric device is activated, causing a first side of the thermoelectric device (e.g., along or near the first substrate) to heat and the second side of the thermoelectric device (e.g., along or near the second, opposite substrate) to cool. Such a heating or cooling effect can be reversed (e.g., wherein the first side is cooled and the second side is heated) by reversing the electrical current through the thermoelectric device. In addition, the amount of heating and/or cooling desired can be regulated by modifying the duty cycle and/or voltage of the thermoelectric device. Accordingly, thermoelectric devices can be used to selectively heat or cool an adjacent surface.

Additional details regarding thermoelectric devices, convective heaters and/or other thermal conditioning devices or systems are provided in U.S. patent application Ser. No. 11/546,928, filed on Oct. 12, 2006 and published as U.S. Publ. No. 2008/0087316 on Apr. 17, 2008; U.S. patent application Ser. No. 11/833,892, filed on Aug. 3, 2007 and issued as U.S. Pat. No. 8,222,511 on Jul. 17, 2012; U.S. patent application Ser. No. 11/972,544, filed on Jan. 10, 2008 and published as U.S. Publ. No. 2008/0173022 on Jul. 24, 2008; U.S. patent application Ser. No. 12/049,120, filed on Mar. 14, 2008 and issued as U.S. Pat. No. 8,143,554 on Mar. 27, 2012; and U.S. patent application Ser. No. 12/695,602, filed on Jan. 28, 2010 and published as U.S. Publ. No. 2010/0193498 on Aug. 5, 2010, all of which are hereby incorporated by reference herein and made a part of the present application.

Thermal conditioning using thermoelectric devices can be accomplished via conductive heating or cooling, wherein the heat is transferred (e.g., directly) to or from the surface to be conditioned. For example, in conductive conditioning, a thermoelectric device and/or other thermal conditioning device can be placed in contact with and/or in close proximity to the object that will be thermally conditioned (e.g., skin, other anatomical location, a surface or other portion of an electronic device or other inanimate object, etc.). Once the thermal conditioning device is activated, the portion of the thermal conditioning device adjacent the target surface (e.g., a first or second side of a thermoelectric device) can be cooled or heated. The heating or cooling of the thermoelectric device and/or any other thermal conditioning device can cause the transfer of heat either away or to the surface to be thermally conditioned (e.g., a subject's skin or other anatomical area, a surface or portion of a device, etc.).

In other embodiments, the heat transfer between a thermal conditioning device (e.g., a thermoelectric device) and a subject's skin or other surface targeted for cooling or heating can be accomplished convectively. For example, air or other fluid can be delivered past or near a heated or cooled portion of a thermal conditioning device to selectively heat or cool such air or other fluid. The heated or cooled fluid can then be transferred to a subject's skin or other target surface. For any of the embodiments disclosed herein, such convective thermal conditioning of skin can be performed either in addition to or in lieu of conductive techniques. For example, in some embodiments, a combination of conductive and convective thermal conditioning is used to selectively cool (or heat) a person's skin, other tissue and/or other anatomical location.

With respect to either conductive or convective cooling or heating of skin (or any other target surface), such thermal conditioning can be performed either directly or indirectly relative to skin (or other surface or portion of a subject). For example, in conductive arrangements, a cooled or heated surface of a thermoelectric device or other thermal conditioning device can be placed either in direct contact (e.g., partially or fully) with the subject's skin or in very close proximity to it (e.g., without the use of any intermediate layers, membranes or other devices or components). Similarly, for devices and systems that utilize convective cooling or heating, cooled or heated air or other fluid can be delivered directly or indirectly (e.g., via one or more intermediate layers, members or the like) to the skin.

In some embodiments, one or more thermoelectric devices and/or other thermal conditioning devices are positioned along, adjacent or near an intermediate layer or device. For example, a thermal conditioning device can be positioned along an exterior surface and/or interior surface of a glove, sleeve, brace, adhesive strip or layer, inflatable member or article of clothing (e.g., shirt, pants, shorts, helmet, hat, etc.). In other configurations, one or more thermal conditioning devices are embedded within an intermediate layer and one or more other layers (e.g., interior layers, exterior layers, etc.), as desired or required.

In embodiments that incorporate one or more intermediate layers, such intermediate layers can comprise one or more materials, such as, for example, natural and/or synthetic fabric, neoprene or other rubber-based materials, other thermoplastics and/or the like. In some embodiments, intermediate layers are sized, shaped and/or otherwise configured to facilitate heat transfer between the thermal conditioning device(s) and the subject's skin (or other target surface). For example, the one or more intermediate layers can comprise a structure and/or thickness that generally promote the transfer of heat either toward or away from the thermal conditioning device.

One embodiment of a thermal conditioning system 2 for selectively cooling or heating the skin and/or other portions of a subject S is schematically illustrated in FIG. 1. In the depicted embodiment, the skin surface being treated (e.g., cooled) is located along a limb (e.g., arm, leg, etc.) of the subject S, which in the illustrated embodiment is illustrated as a cylinder for convenience. However, the thermal conditioning system or components thereof can be used to cool and/or heat any other portion of the subject's skin or anatomy (e.g., torso, head, etc.).

With continued reference to FIG. 1, a plurality of the thermal conditioning assemblies 10 can be strategically positioned along the skin of the subject. In the depicted embodiment, a total of nine assemblies 10 have been positioned along various portions of the subject's limb in order to cool adjacent portions of the subject skin. However, in other embodiments, fewer (e.g., 1, 2, 3, 4, 5, 6, 7, 8) or more (e.g., 10, 11, 12, 13, 14, 15, more than 15, etc.) assemblies 10 can be used, as desired or required for a particular application, treatment protocol or use. For example, the exact number of thermal conditioning assemblies that are required or desired can depend on one or more factors, such as, for example, the size of the individual assemblies, the cooling or heating capacity of the assemblies, the size, type and other details of the subject's target skin or other anatomical location, the amount of cooling or heating that is required and/or the like.

In the embodiment illustrated in FIG. 1, the assemblies 10 are located in an aligned or substantially aligned orientation along a longitudinal axis of the subject's limb. As shown, the assemblies 10 are located along three distinct rows, with each row having a total of three assemblies 10. In other embodiments, the orientation, quantity, type, spacing, the inclusion of additional components or features (e.g., sensors, intermediate layers, etc.) and/or other details related to the thermal assemblies 10 of a skin conditioning system 2 can be different than illustrated in FIG. 1 and/or otherwise disclosed herein. For example, to further clarify the point that great variations in design can exist between various embodiments, in some configurations, a skin conditioning system can comprise only a single thermal conditioning assembly. The terms thermal conditioning assembly, thermal conditioning device, thermal assembly and cooling assembly are used interchangeably herein.

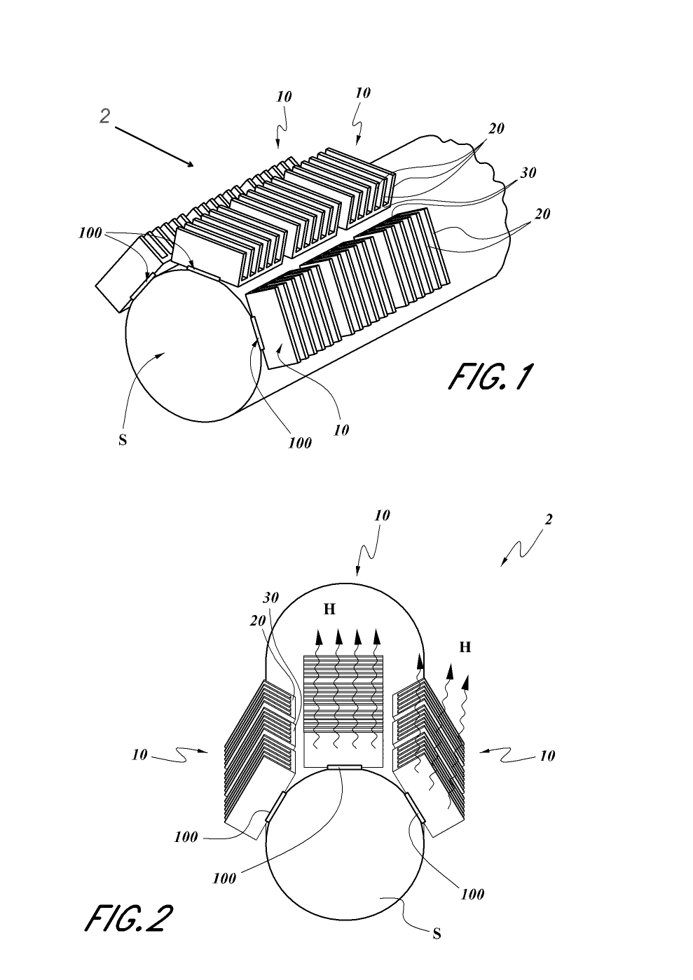

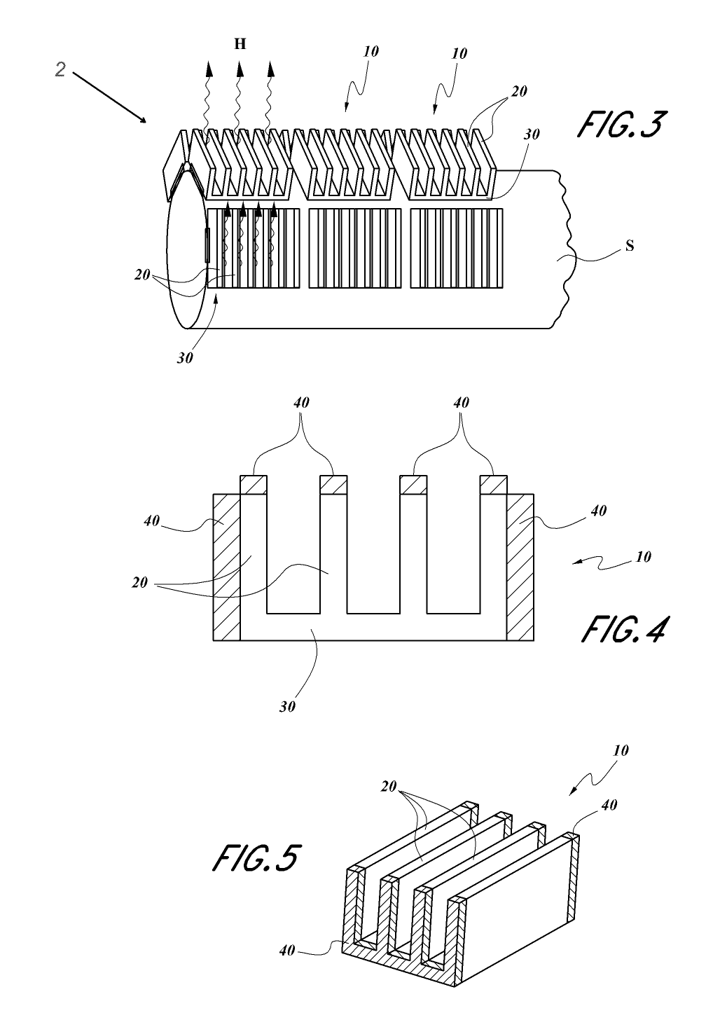

As illustrated in FIG. 1, as well as the related views in FIGS. 2 and 3, a thermal conditioning assembly 10 can comprise a heat sink 20 that extend from a base 30. The fins or other heat transfer members 20 can form a single, unitary structure with the base 30. Alternatively, the heat sink (e.g., heat transfer members) 20 can be separate from the base (and/or each other), as desired or required. Accordingly, one or more portions of the thermal assembly 10 can be attached (and/or otherwise positioned adjacent) to one another using one or more connection devices, methods and/or features, such as, for example, adhesives, mechanical or other types of fasteners (e.g., screws, rivets, clips, etc.), pressure or friction fit connections and/or the like. As used herein, the term “heat sink” should be given its ordinary meaning and may include, without limitation, one or more fins, pins and/or other heat transfer members or portions. For example, in some embodiments, the heat sink 20 includes a base 30. In other embodiments, the heat sink 20 is separate and distinct, partially or completely, from the base 30 (e.g., structurally, thermally, etc.).

One or more thermoelectric devices or other thermal conditioning devices 100 (e.g., other cooling or heating devices, convective heaters, etc.) can be positioned along, within or near the heat transfer members 20 and/or the base 30 of the assembly 10. For example, in the depicted embodiment, one or more thermoelectric devices 100 are located along the base 30 (e.g., above and/or below the base) of the thermal assembly 10. Accordingly, in some embodiments, when activated, the lower surface of the thermal conditioning device 100 conductively cools (or heats, as desired or required) by transferring heat away (or to) the skin of the subject S. In arrangements where the thermal conditioning device 100 comprises a thermoelectric device (and/or other thermal conditioning device), a waste side that is generally opposite of the main side being cooled, may be heated (or vice versa).

Thus, in such embodiments, when the thermal conditioning device 100 is activated and the subject's skin is being selectively cooled by the thermal conditioning system 2, heat is being produced along the opposite (e.g., top) side of the conditioning device 100. Accordingly, the heat exchange members (e.g., fins) 20 of the assembly 10 can help transfer that waste heat away from the system 2 and the subject S. As shown, the heat exchange members 20 can include a plurality of spaced apart fins or portions (e.g., plates, pins, sheets, etc.). In some embodiments, such heat exchange members 20 comprise one or more high heat transfer materials and/or other materials with favorable thermal conductive properties (e.g., copper, aluminum, etc.), a relatively large surface area, relatively thin profile and/or one or more other features for promoting more efficient heat transfer between the surrounding environment (e.g., ambient air) and the heat being produced by the thermoelectric device or other thermal conditioning device 100. In some embodiments, in order to further enhance the transfer of waste heat away from the thermal assembly 10 and the subject's skin, one or more fluid transfer devices (e.g., blowers, fans, pumps, etc.) can be used to deliver air through and/or near the heat transfer members 20, as desired or required.

As illustrated in FIGS. 2 and 3, during use, heat H can pass through the heat transfer members 20 (e.g., fins, pins, etc.) and be carried away from the electrically activated thermal assemblies 10. Meanwhile, the side of the thermal conditioning device (e.g., thermoelectric device) 100 adjacent the subject can conductively cool the subject's skin. With reference to the embodiment of the system 2 illustrated in FIGS. 1-3, each thermal conditioning assembly 10 can include one or more (e.g., 2, 3, 4, 5, 6, 7, 8, 9, 10, more than 10, etc.) thermal conditioning devices 100, as desired or required. As noted above, the thermal conditioning device 100 can be located along any location of a thermal assembly, such as, for example, without limitation, at, along or near the bottom of the assembly (e.g., along an upper and/or lower surface of a base 30, at least partially within a base or other portion of the assembly, etc.), along a side of an assembly (e.g., at or near one of the fins or other heat transfer members, etc.) and/or at or near any other location of the assembly. Accordingly, depending on the exact locations of the thermal conditioning device or devices 100, one or more thermal conditioning devices 100 (e.g., thermoelectric devices) of the assembly can be in direct or substantially direct contact with the subject's skin. Such contact can be continuous or partial (e.g., intermittent).

Further, in some embodiments, the thermal conditioning system 2 can be used to transfer heat from or to the subject's skin (e.g., to selectively cool or heat the subject's skin) without direct contact between the cooled or heated surface(s) of a thermal conditioning device 100. For example, the thermoelectric device or other thermal conditioning device 100 can be a particular distance away from the adjacent skin surface. In some embodiments, such a clearance distance can be 1 mm to 20 mm (e.g., 1, 2, 3, 4, 5, 10, 15, 20 mm, values between the foregoing, etc.). In other embodiments, the clearance is less than about 1 mm (e.g., 0.01, 0.05, 0.1, 0.2, 0.3, 0.4, 0.5, 0.6, 0.7, 0.9, 0.9 mm, values between the foregoing, etc.) or more than about 20 mm (e.g., 21, 22, 25, 30, 35, 40, 50, 60 mm, more than 60 mm, values between the foregoing, etc.). In some embodiments, the system can be configured to selectively vary the clearance distance between the thermal conditioning device 100 and the adjacent skin surface, based on, for example, a particular feedback control routine (e.g., closed loop system), another operational scheme and/or the like.

In some embodiments, as discussed herein, the subject's skin can be selectively cooled and/or heated using a convective cooling and/or heating device, either in lieu of or in addition to conductive heating techniques. For example, in some embodiments, cooled and/or heated air can be delivered to a targeted skin area to thermally condition it. Convective thermal conditioning can use one or more fluid transfer devices (e.g., blowers, fans, pumps, etc.), ducts or other fluid conduits and/or any other component to help transfer fluids through the necessary fluid path.

The view of FIG. 3, schematically illustrates a side view of a plurality of thermal conditioning assemblies 10 aligned in several rows. As shown, the assemblies 10 can be placed next to each other, in the longitudinal and/or radial direction, as desired or required to cool (or heat) an area of the subject's skin. In embodiments comprising thermoelectric devices or related technology to cool the skin, heat H produced along the opposite side of the thermoelectric device can rise through the heat transfer members 20 and be transferred (e.g., with or without the use of a fan or other fluid transfer device). In some embodiments, air can be passed near, along and/or through the heat transfer members 20 to provide for more enhanced cooling of the thermoelectric devices (or other thermal conditioning devices).

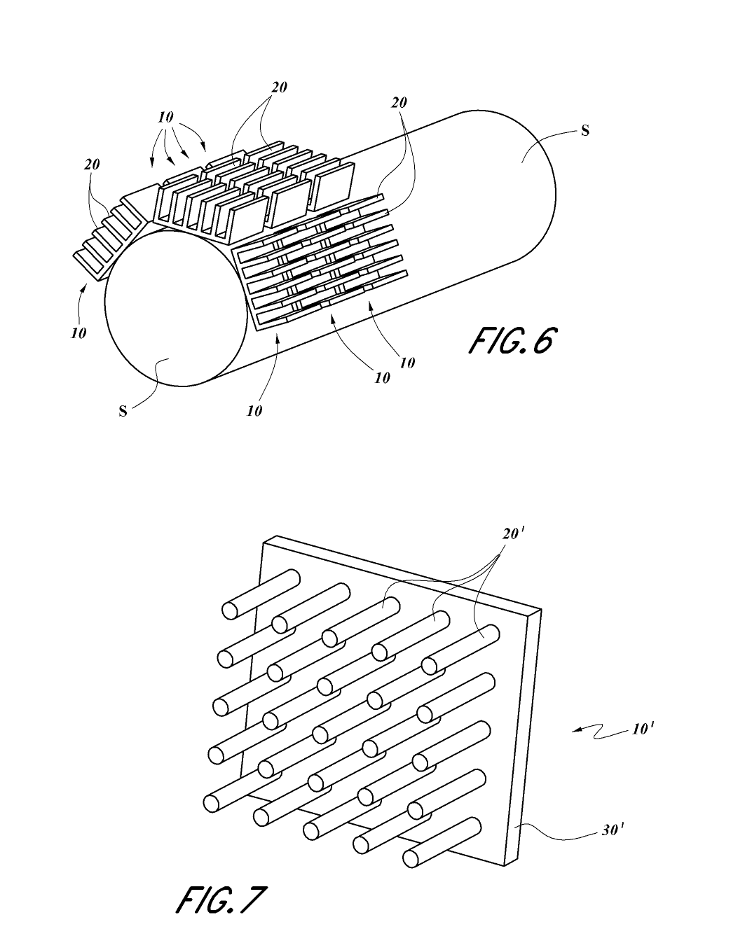

FIGS. 4 and 5 illustrate different views of one embodiment of a thermal conditioning assembly 10 for use in skin cooling or heating systems and applications. As shown, one or more outer surfaces of the assembly 10 can comprise a protective, thermal insulation layer, component or material 40. The use of insulation can help protect a user who comes in contact with the various assemblies against undesirably hot portions of the heat transfer members 20 or other portion of the assembly 10. In some embodiments, the thermal insulation layer or component 40, which can be secured to the assembly using adhesives, mechanical fasters and/or the like, is positioned along the sides of the assembly (e.g., adjacent the heat transfer members 20), along the top of the heat transfer members and/or along any other surface of the assembly 10 that can come in contact with other parts of a user.

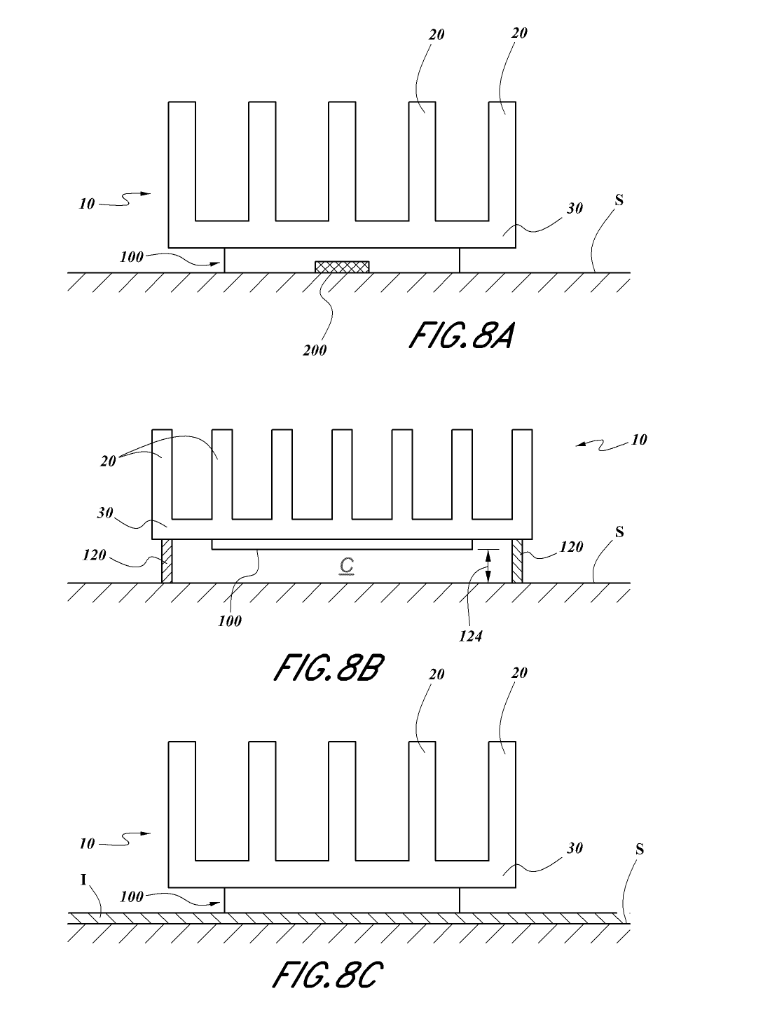

In other embodiments, as schematically illustrated in FIG. 6, the plurality of thermal conditioning assemblies 10 can be arranged so the heat transfer members 20 of longitudinally assemblies 10 align with one another. This is different than the alignment of longitudinally adjacent members 10 of the embodiment illustrated in FIGS. 1-3. The specific arrangement of heat transfer members 20 can be selected to create a desired orientation of heat transfer members 20 (e.g., in relation to one another and/or to the subject).

As illustrated in FIG. 7, in some embodiments, the heat transfer members 20′ of a thermal conditioning assembly can comprise a plurality of pins that extend from a base 30′. As with any other assembly embodiments disclosed herein, the pins, fins and/or other heat transfer members 20, 20′ can form a unitary structure with the base 30, 30′. Alternatively, however, the heat transfer members 20, 20′ can be separate from the base 30, 30′. In such arrangements, the heat transfer members 20, 20′ can be attached to the base 30, 30′ using one or more connection devices, features or methods (e.g., welds, adhesives, screws, rivet, other fasteners, press fit or friction fit connections, etc.). The pins 20′ can comprise a generally cylindrical shape (e.g., as illustrated in FIG. 7). Alternatively, however, the pins can comprise any other cross-sectional shape (e.g., square, rectangular, triangular, other polygonal, oval, irregular, etc.). Further, the overall shape, spacing, quantity, orientation and/or other details regarding the fins 20, pins 20′ and/or other heat transfer members can be different than illustrated herein, as desired or required. As with other embodiments disclosed herein, one or more thermoelectric devices and/or other thermal conditioning devices (not shown) can be positioned on, within and/or near the base 30′ so that, when activated, the thermal conditioning assembly 10′ can selectively cool or heat (e.g., transfer heat away from or to) a subject skin or other target anatomical location.

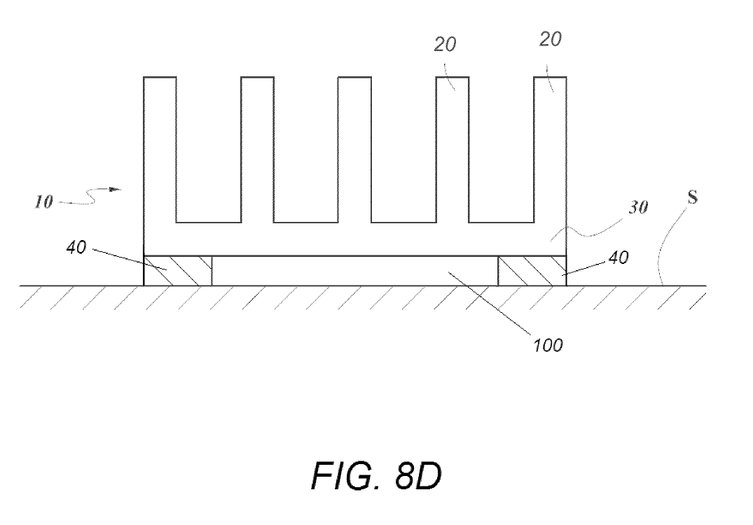

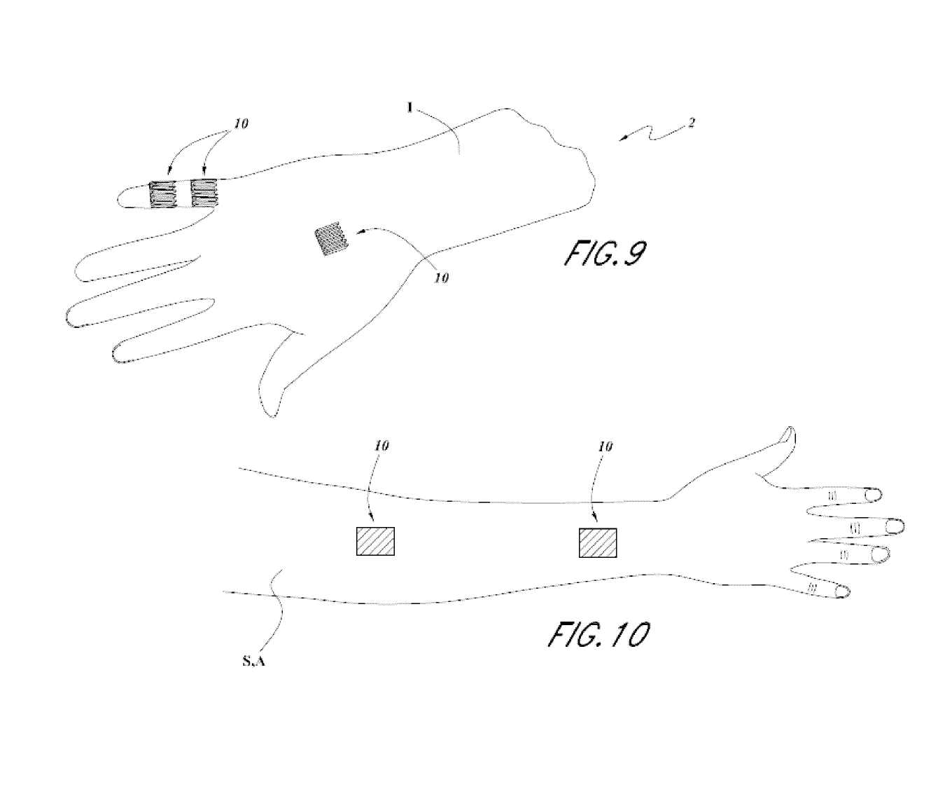

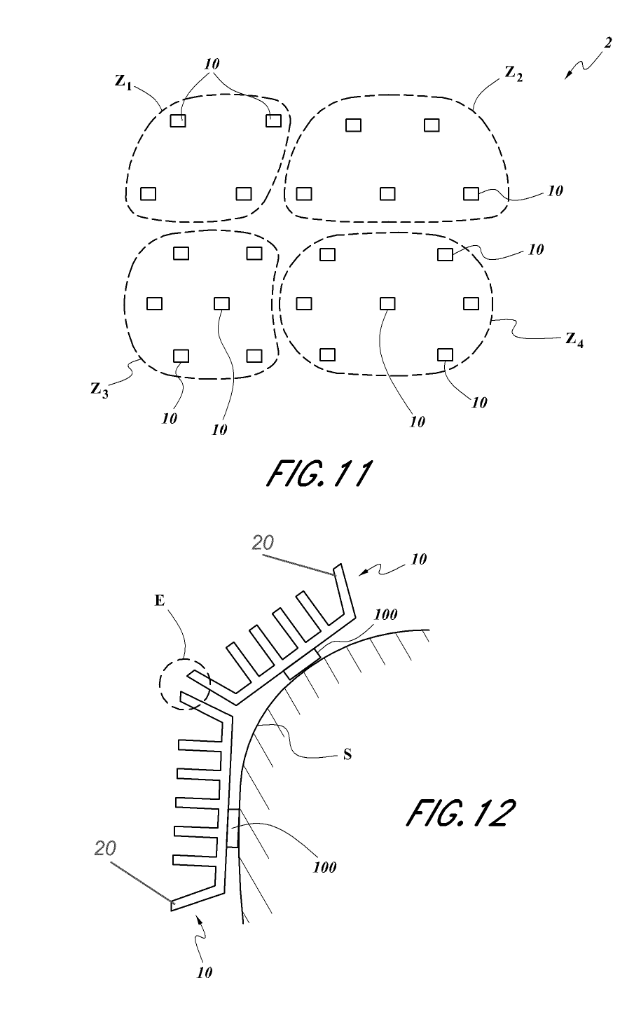

FIG. 8a illustrates one embodiment of a thermal conditioning assembly 10 configured for direct placement along a portion of a subject's skin. As shown, the assembly 10 comprises one or more heat transfer members 20 (e.g., fins, pins, etc.) that extend from a base 30. One or more thermoelectric devices and/or other thermal conditioning devices 100 can be positioned along (e.g., top, bottom, etc.), within, adjacent or near the base 30 (and/or any other portion of the assembly 10). When activated, the thermal conditioning device 100 can selectively cool or heat the adjacent skin surface. For example, during a cooling procedure using the assembly, a thermoelectric device 100 can be placed on or near the target skin surface of the subject. Thus, the thermal conditioning device 100 can contact (e.g., partially or completely) or not contact the subject (e.g., the subject's skin).

According to some embodiments, as illustrated in FIG. 8b, the assembly 10 can comprise one or more spacers 120 that are configured to contact the subject's skin during use of the cooling system. In the illustrated embodiment, the spacers 120 are secured to the bottom of the base 30 of the assembly 10. However, in other embodiments, one or more spacers can be secured to the thermal conditioning device 100 and/or any other portion of the assembly or cooling system, either in lieu of or in addition to the base 30, as desired or required. The spacers 120 can include posts or other protruding members having generally smooth bottom surfaces that engage the subject's skin. In some embodiments, the spacers 120 can include a long continuous lip and/or other generally continuous members that extend along a longer portion (e.g., partially or completely) of the assembly 10. In some embodiments, the spacers 120 comprise one or more rollers and/or other low friction devices or components to assist in moving or repositioning the assembly along the subject's skin. As shown, the spacers 120 can help maintain a particular clearance 124 between the subject's skin (or other anatomical surface or area of the subject being thermally conditioned) and the thermal conditioning device 100. For example, as noted above, in some embodiments, the clearance 124 is 1 mm to 20 mm (e.g., 1, 2, 3, 4, 5, 10, 15, 20 mm, values between the foregoing, etc.). In other embodiments, the clearance is less than about 1 mm (e.g., 0.01, 0.05, 0.1, 0.2, 0.3, 0.4, 0.5, 0.6, 0.7, 0.9, 0.9 mm, values between the foregoing, etc.) or more than about 20 mm (e.g., 21, 22, 25, 30, 35, 40, 50, 60 mm, more than 60 mm, values between the foregoing, etc.). As discussed in greater detail herein, the spacers can provide an intermittent or continuous interface between a thermal conditioning device (e.g., a thermoelectric device) and the targeted portion of a subject's anatomy (e.g., skin surface). For example, the spacers or other intermediate layer or member can extend continuously or substantially continuously between the thermal conditioning device and the skin. In other embodiments, however, as illustrated in FIG. 8b, the spacers or other intermediate layers or members are located along only certain portions between the thermal conditioning device and the skin (e.g., they do not extend continuously and/or completely between the thermal conditioning device and the skin or other targeted region of the subject's anatomy). The clearance can create a cavity C which can be pressurized or within which a vacuum can be created.

With any of the embodiments disclosed herein, the various cooling or other conditioning system can include one or more sensors. For example, as illustrated in FIG. 8a, a thermocouple, thermistor or other temperature sensor 200 can be positioned adjacent the thermoelectric device 100 of the thermal conditioning assembly 10 and/or the subject's skin. Such sensors can help ensure that the level of cooling (and/or heating, if heating of the subject is desired) is maintained within particular limits. This can help to prevent or reduce the likelihood of potentially harmful and/or otherwise damaging temperature extremes during use of the system. In the event a detected temperature (e.g., at or near the thermal conditioning device, along another part of the device, at or near the subject skin or other targeted portion of the subject's anatomy, etc.) is determined to be too high or too low, one or more operational properties of one or more of the thermoelectric devices 100 can be modified accordingly, either manually or automatically (e.g., based, at least in part, on a closed-loop control system or other operational scheme). For example, in some embodiments, if it is determined that a temperature of the thermal conditioning system and/or the subject's skin is above or below a particular threshold, power to one or more of the system's thermal conditioning devices (e.g., thermoelectric device, other heating or cooling devices, etc.) can be modified (e.g., reduced or increased) for safety, efficacy, comfort and/or other reasons. For example, such control schemes can help one or more components of the thermal conditioning assembly 10. Additionally, the use of such sensors 200 can help to prevent or reduce the likelihood of potentially harmful and/or otherwise damaging sustained temperatures to the user of the thermal conditioning assembly 10 (e.g., extended periods of low and/or high temperatures).

Such sensors 200 can be positioned along one or more thermal conditioning assemblies 10 and/or any other component, device and/or portion of the cooling system. Sensors can include temperature sensors, heat flux sensors, humidity sensors, condensation, moisture sensors, ion or chemical sensors and/or any other type of sensor, as desired or required. Other examples of sensors 200 include physiological sensors such as heart rate sensors, blood-oxygen sensors, perfusion sensors and other types of circulatory sensors, galvanic (skin conductance) sensors as well as motion sensors and pressure sensors. In various embodiments, the sensors 200 can be positioned in remote areas, or in other words, in areas of the user away from the thermal conditioning assemblies 10 or areas where thermal conditioning assemblies 10 are not located.

For any of the skin conditioning systems disclosed herein, one or more intermediate layers or components can be positioned adjacent the thermal conditioning assemblies 10 and/or other components of a system. For example, as illustrated in FIG. 8c, one or more intermediate layers I, such as, for example, at least a portion of a glove, sleeve, brace, adhesive strip or layer, fabric, article of clothing (e.g., shirt, pants, shorts, helmet, hat, etc.), bladder (e.g., balloon) or other expandable member, conductive slurry, flexible band, fibrous pad and/or the like can be positioned between a thermal conditioning device 100 (or, e.g., a thermal conditioning assembly 10 to which the device 100 is secured) and the subject's skin being thermally conditioned. Such intermediate layers or components I can help prevent direct contact between the subject's skin and the various assemblies and device of a cooling system. In some embodiments, intermediate layers or components can be reusable (e.g., via sterilization, other cleaning, etc.) or disposable (e.g., replaceable with a new layer I), as desired or required. In some embodiments, such intermediate layers or components I can help distribute the transfer of heat more evenly between the subject (e.g., skin surface, other portion of the subject's anatomy, etc.) and the one or more thermal conditioning devices (e.g., thermoelectric devices). Accordingly, in some embodiments, the intermediate layers or components can include relatively favorable thermal conductivity properties.

As discussed herein with reference to FIGS. 4 and 5, a thermal conditioning assembly 10 of the system can comprise one or more protective, thermal insulation layers, components or materials 40. FIG. 8d illustrates one embodiment of a thermal conditioning assembly 10 comprising one or more layers of a thermal insulation layer 40 at least partially surrounding the thermoelectric device or other thermal conditioning device 100. Such a configuration can help shield the subject or other user of the system from the thermal conditioning device 100.

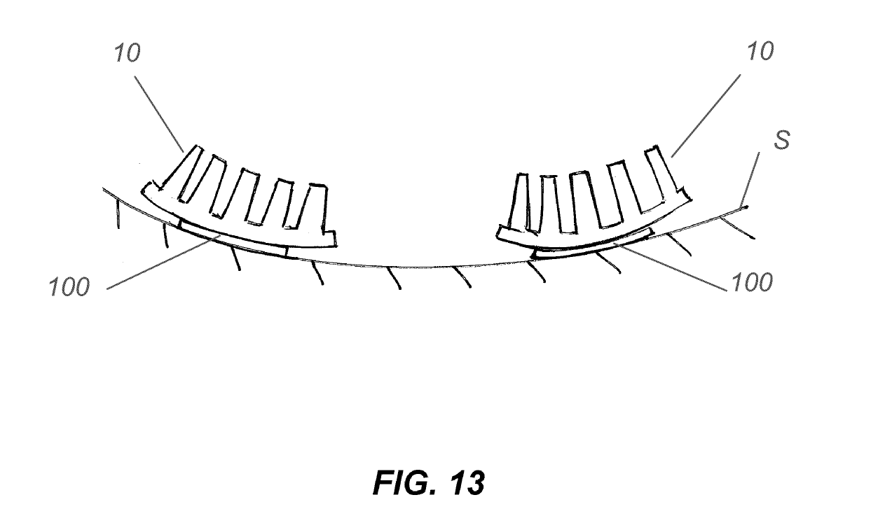

One embodiment of a skin conditioning system 2 in the shape of a glove is illustrated in FIG. 9. As shown, the system can include one or more intermediate layers or components I that are shaped, sized and otherwise configured to be worn (e.g., like a glove). Alternatively, the system 2 can include a glove-shaped intermediate layer I that is configured to simply be placed over a subject's hand. The system 2 can include any other shape to match (or approximate) the corresponding portion of the subject's anatomy being cooled or otherwise thermally conditioned. For example, a system 2 can include one or more intermediate layers I (e.g., positioned between one or more thermal conditioning assemblies 10 and the subject) that are in the shape of (or configured to be worn on), at least in part, a foot, a leg, an arm, a hand, a torso, a neck, a head, etc.). As shown in FIG. 9, the system 2 can include one or more (e.g., 2, 3, 4, 5, more than 5, etc.) thermal conditioning assemblies 10 placed along at least a portion of the intermediate layer I.

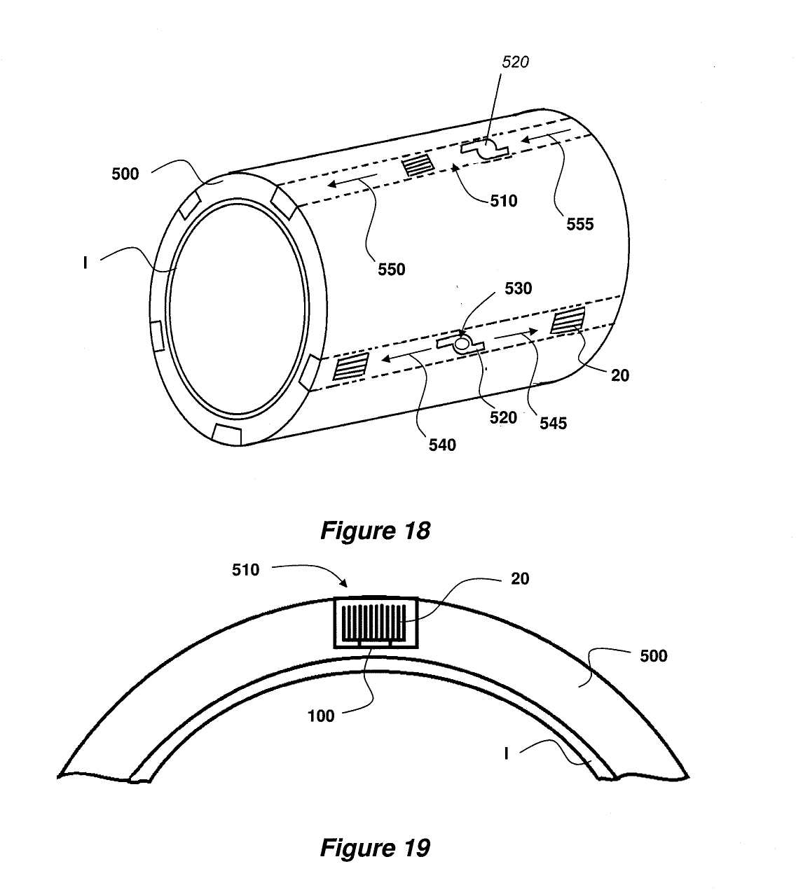

As discussed herein, a thermal conditioning assembly 10 can be positioned directly on the subject's skin S. For example, in the embodiment illustrated in FIG. 10, two separate assemblies 10 have been positioned along a subject's arm A. In other arrangements, the quantity, spacing, size and/or other details the thermal conditioning assemblies 10 included in a particular system 2 can vary, as desired or required. In any of the embodiments disclosed herein, the various assemblies 10 and/or other electrical components of a system can comprise one or more of the following: a power supply (e.g., battery, connections to a central or main power source, a controller, a sensor and/or the like). For example, the battery can be integrated with the system 2 such as within an interior layer I or exterior layer (e.g., exterior layer 500, FIGS. 18 and 19). The system can also include an integrated power adaptor connection, such as, for example, within an interior layer I, exterior layer 500 and/or other portion of the system. In some embodiments, the battery or battery assembly that provides power to the thermal conditioning system can be a rechargeable or non-rechargeable, as desired or required. In some embodiments, as discussed, power can be supplied to one or more of the electrical components of the thermal conditioning system using a hardwired connection (e.g., AC or DC) and/or any other power device or method (e.g., solar panel). Where a temperature sensor is included in a particular system, such a sensor can be configured, in some embodiments, to automatically disable a thermal conditioning device 100 (e.g., a thermoelectric device) when the sensor reaches a particular temperature. Such sensors can be incorporated into any of the embodiments disclosed herein.

FIG. 11 illustrates one embodiment of a cooling or other thermal conditioning system 2 comprising a plurality of thermal conditioning zones Z₁, Z₂, Z₃, Z₄. In the illustrated embodiment, the system 2 comprises a total of four zones. However, in other arrangements, more (e.g., 5, 6, 7, 8, 9, 10, more than 10, etc.) or fewer (e.g., 1, 2, 3) zones can be included, as desired or required. For example, each zone can be associated with particular sub-portions of the subject's anatomical region being cooled (e.g., if the system is used for cooling the subject's arm, the various zones can be aligned with, e.g., the upper arm or shoulder, elbow, forearm, hand, etc.). In addition, each zone Z₁, Z₂, Z₃, Z₄ can include one or more thermal conditioning assemblies 10, in accordance with the various embodiments disclosed herein. Regardless of the exact layout and configuration of the zones, and the various assemblies and/or other components 10 included therein, the use of separate zones can enable for various customized temperature or thermal transfer profiles (or other thermal conditioning, e.g., cooling, heating) along the portion of the subject's body along which the system 2 is placed. In some embodiments, a thermal profile can be designed to create a desired response in one or more thermal receptors (e.g., hot/cold receptors within the subject's body).

In some embodiments, the zones created by an array of thermal conditioning assemblies can be independently controllable to provide more precise control of the resulting temperature profile on the skin surface or other portion of the subject's anatomy on which the system is being used. In some embodiments, a zone includes only a single thermal conditioning assembly 10. However, in other embodiments, a zone can include two or more thermal conditioning assemblies, as desired or required. As noted herein, each thermal conditioning assembly can include one or more thermal conditioning devices (e.g., thermoelectric device). Thus, in some embodiments, a zone can include only a single thermal conditioning device or multiple conditioning devices, as desired or required for a particular design.

According to some embodiments, the temperature profile created by an array of individually controllable thermal conditioning assemblies (and the zones in which they are located) can balance temperatures and/or thermal transfer rates along the skin surface. For example, a temperature profile can be chosen to allow for a smoother transition in temperatures from a first area (e.g., zone) to a second area (e.g., zone) of a skin surface. However, in other embodiments, the temperature variations between adjacent conditioning zones or areas created by an array of individually controllable thermal conditioning assemblies can be non-smooth (e.g., abrupt). In some embodiments, the level of cooling (or heating) between adjacent zones varies between 5 and 20° C. (e.g., 5-6, 6-7, 7-8, 8-9, 9-10, 10-11, 11-12, 12-13, 13-14, 14-15° C., temperatures between the foregoing ranges, etc.). In other embodiments, the level of cooling (or heating) between adjacent zones varies between 0 and 5° C. (0-1, 1-2, 2-3, 3-4, 4-5° C., temperatures between the foregoing ranges, etc.) or varies by more than 15° C. (15-20, 20-25, 25-30, 30-40, 40-50° C., greater than 50° C., etc.), as desired or required. However, as noted above, the temperature variations between adjacent conditioning zones or areas created by an array of individually controllable thermal conditioning assemblies can be smooth. Accordingly, in some embodiments, the temperature variations discussed above can occur between zones that are not adjacent to one another (e.g., zones that are located at opposite ends of the array). In some embodiments, the use of an array of thermal conditioning devices that create separate conditioning zones can create a linear temperature (e.g., thermal conditioning) profile along a section of the subject's skin or other anatomical area being targeted. For example, adjacent zones can be operated at increasingly higher or lower temperatures so that the entire system exhibits a linear or generally linear thermal treatment profile. In other embodiments, the temperature profile along a system, and thus the portion of the subject's skin being cooled/heated, can be non-linear. For example, adjacent zones or areas of the system can alternate between higher and lower levels of thermal conditioning. In some embodiments, the thermal or temperature profile along at least a portion of the thermal conditioning system is sinusoidal, logarithmic, step-like (e.g., alternating higher and lower temperatures), irregular and/or the like.

A temperature profile created by an array of individually controllable thermal conditioning assemblies can be used to provide greater or lesser degrees of heating or cooling to certain body parts which may be more or less sensitive to temperature. In some embodiments, the use of zones or arrays of thermal conditioning assemblies 10 can beneficially provide more precise targeting of thermal conditioning. As noted herein, the various thermal conditioning assemblies in an array can be individually controlled. For example, in some embodiments, the thermal conditioning assemblies are provided to the user with specific thermal conditioning characteristics that are fixed. For example, each of the thermal conditioning assemblies included in an array can be fixed to a particular level of heating or cooling to create a desired thermal conditioning profile and pattern for that system. In other embodiments, however, the level of cooling or heating one or more (e.g., all) of the thermal conditioning assemblies included in an array can be adjusted by the subject or user and/or the doctor or other care giver using the system. For example, the specific operating temperature of an assembly along the subject's skin can be selected. In other embodiments, a general setting of thermal conditioning can be selected (e.g., low-medium-high, different levels, etc.), as desired or required. In yet other embodiments, a relative level of cooling or heating between adjacent zones created by the plurality of thermal conditioning assemblies can be selected. Such an operational scheme can be aided, in certain arrangements, with the assistance of one or more temperature sensors.

For embodiments that permit users to adjust an operational parameter of a thermal conditioning assembly, such adjustment can be accomplished using one or more controllers (e.g., switches, buttons, dials, touchscreens, etc.) positioned on, near or adjacent the thermal conditioning assemblies. For example, the controllers can be located on or within a portion of the assemblies. Alternatively, the controllers can be located in a separate control unit (e.g., separate module, remote controller, etc.) that is operatively coupled (e.g., wirelessly, using a wired connection, etc.) to the thermal assemblies and the overall system. In still other embodiments, the thermal conditioning assemblies, and the array and zones which they help create, can be controlled using a computer, a smartphone, a tablet and/or another computing device. As discussed in greater detail herein, the various thermal conditioning devices (e.g., thermoelectric devices) that are included in an array of thermal conditioning assemblies can connected to each other in parallel, series or a combination thereof.

In some embodiments, the plurality of zones can be selectively activated in sequences to provide additional therapeutic benefit to the user. Such selective activation can be chosen by the user or form part of a programmed cycle in the thermal conditioning system. For example, in some embodiments, the system can alternately activate and/or deactivate certain zones and the thermal conditioning assemblies located therein. For example, at a first point in time, a first zone Z₁ can be deactivated while one or more other zones Z₂, Z₃, Z₄ remain activated. Further, at a second point in time a different zone Z₂ can be deactivated, while previously deactivated zones (e.g., Z₁) can be reactivated and previously activated zones (e.g., Z₃, Z₄) remain activated. In such an example, at a third point in time, a third zone (e.g., Z₃) can be deactivated, while the previously deactivated zone (e.g., Z₂) is reactivated and the other previously activated zones (e.g., Z₁, Z₄) remain activated. Continuing with the example embodiment, at a fourth point in time, a fourth zone (e.g., Z₄) can be deactivated, while the previously deactivated zone (e.g., Z₃) is reactivated and the other previously activated zones (e.g., Z₂, Z₄) remain activated. The process can then repeat or a different sequence can be used. As used herein, the term deactivated is broad and can refer to completely turning off the thermal conditioning assemblies located within a zone or simply reducing the power or duty cycle (and thus the level of thermal conditioning) created by the thermal conditioning assemblies. Thus, the zones need not be fully turned off during a deactivation. Similarly, activation of a zone (and the thermal conditioning assemblies located therein) can include turning on the thermal conditioning devices (e.g., thermoelectric devices) located therein to a power level or duty cycle that is lower than a maximum. For example, rather than deactivating a zone, the system can increase or decrease the amount of power being input into a corresponding thermoelectric device or other thermal conditioning device of the assembly.

In some embodiments, the use of sequencing and the resulting temperature variations it helps create can provide one or more benefits and advantages. For example, the resulting temperature variations can, in certain arrangements, provide additional therapeutic benefit to the subject. Moreover, the use of sequencing can help avoid or reduce the likelihood of potentially harmful levels of thermal conditioning to the skin surface or other targeted portion of the subject's anatomy. In some embodiments, adjacent thermal conditioning assemblies (or adjacent zones that include one or more thermal conditioning assemblies) are operated in different modes. For example, one thermal conditioning assembly (or zone) is cooling the targeted skin surface or other anatomical area of the subject, while an adjacent assembly (or zone) is heating the skin or area. In other embodiments, both of the adjacent assemblies or zones cool or heat, but at varying levels.

In some embodiments, due to the non-linear (e.g., curved, convex, irregular, etc.) shape of various regions of the anatomy of a subject, systems can be customized to a particular shape. For example, as illustrated in FIG. 12, heat sinks 20 of adjacent assemblies 10 can be angled along the ends E to accommodate the curvature of the body portion being cooled (e.g., arm, leg, other limb, etc.). Likewise, as shown in FIG. 13, one or more assemblies 10 (e.g., heat sinks) and/or other portions of the conditioning system can include a curvature to accommodate the shape of the subject's skin being cooled or heated.

In some embodiments, one or more membranes, layers and/or other devices are placed over and/or around the targeted skin portion of a subject before commencing a cooling or heating procedure, either in addition to or in lieu of including an intermediate layer or other layer in the thermal conditioning system itself. For example, in some arrangements, one or more thin layers of plastic (e.g., vinylidene chloride or saran wrap, polyethylene, other polymeric materials, etc.), can be positioned along or around an arm, leg, foot, neck and/or any other anatomical portion before applying the thermal conditioning system to the subject's skin.

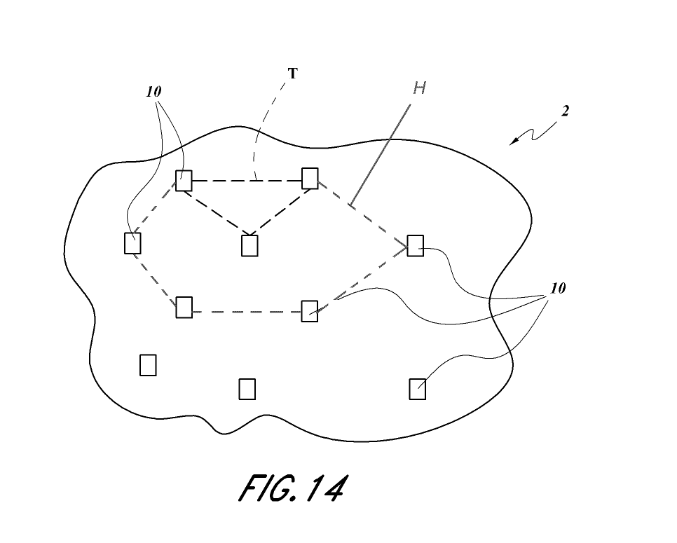

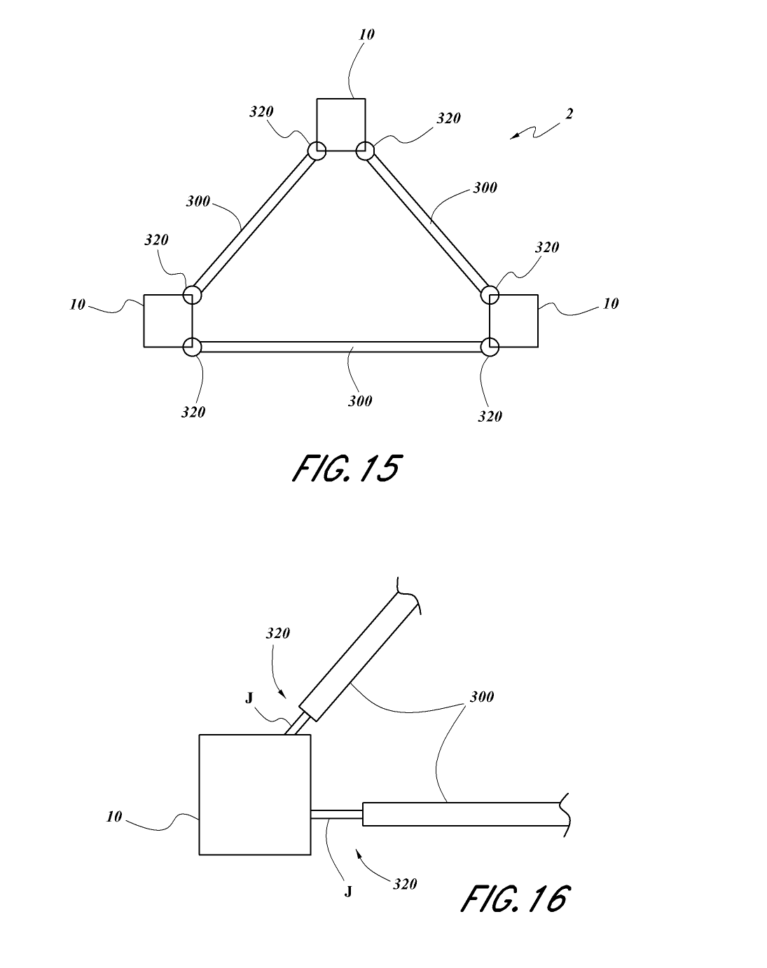

In any of the embodiments disclosed herein, the various heat transfer assemblies 10 (e.g., heat sinks and thermal conditioning devices) of a system 2 can be arranged in a hexagonal arrangement H, as illustrated schematically in FIG. 14. Such an arrangement can provide more uniform thermal coverage. However, in other embodiments, a different layout or arrangement can be used, such as, for example, rectangular (e.g., grid), circular or oval (e.g., plurality of concentric circles or ovals), etc., as desired or required.

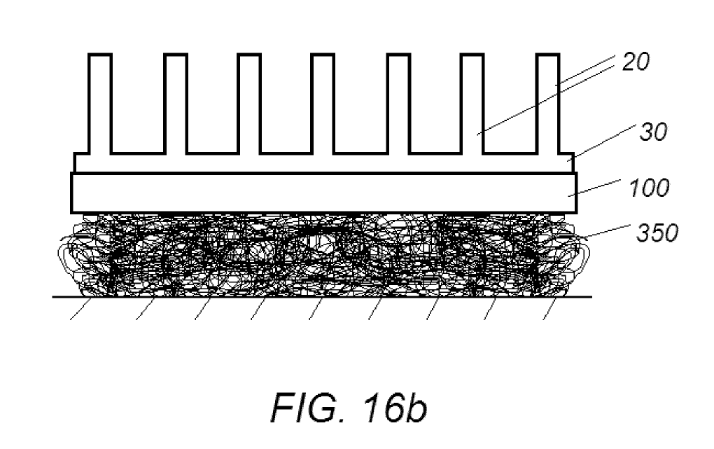

As noted herein, portions of the system 2 can also be configured to at least partially flex or bend to generally conform to the subject's anatomy. Thus, a hexagonal arrangement, defining triangularly-shaped panels T (FIG. 14) between adjacent assemblies 10, can provide for a more enhanced flexibility of the system 2. As illustrated in FIG. 15, in any of the embodiments disclosed herein, adjacent assemblies 10 of a system 2 can include one or more interconnecting members 300 that connect two or more of the assemblies 10 to each other. Such interconnecting members 300 can mechanically, thermally, and/or electrically couple adjacent assemblies 10 to each other, and may include, without limitation, one or more rigid, semi-rigid and/or flexible materials or components (e.g., metals, alloys, plastic, etc.). In some embodiments, for example, the interconnecting members 300 comprise tubes (e.g., solid or hollow) that include a circular, oval, rectangular, triangular, other polygonal and/or any other cross sectional shape. One or more wires or other electrical conductors can be routed through hollow embodiments of the interconnecting members 300. This can provide a convenient manner in which to electrically couple the various assemblies 10 to each other and/or to a power supply or control module. Moreover, in some embodiments, the interconnecting members 300 can comprise thermally conductive materials. Thus, the interconnecting members 300 can serve as heat pipes. These interconnecting members 300 can be thermally coupled to the main side of the thermoelectric devices 100. Accordingly, a temperature gradient of main side temperatures can be formed between the interconnected assemblies 10 which can advantageously create a smoother temperature distribution throughout a particular skin or other anatomical conditioning system. In some embodiments, the interconnecting members 300 can be at least partially thermally coupled with the waste side of the thermoelectric devices such as the heat sink. With continued reference to FIG. 15, the ends 320 of the interconnecting members 300 can comprise rigid or movable connections to the corresponding assemblies 10. For example, as illustrated in FIG. 16, in some embodiments, the end 320 can include a flexible joint or other rotatable features J to improve the overall flexibility of the system 2.

In any of the embodiments disclosed herein, the thermal conditioning system 2 can include assemblies that only comprise a thermal conditioning device 100 (e.g., thermoelectric device, other cooling or heating element, etc.) without the need for additional portions or features (e.g., heat sink).

As discussed herein, the system can include one or more intermediate or other base layers onto which the various thermal conditioning devices 100 or assemblies 10 are positioned. Such a layer can comprise a flexible structure or features so that the system can generally conform to the subject's anatomy. For example, in some arrangements, the thermal conditioning components (e.g., devices, assemblies, etc.) are secured to a vinyl sheet or other fabric or layer (e.g., thermally conductive pad or mat, slurries, bladders or other expandable members, etc.).

As schematically illustrated in FIG. 16b, in some embodiments, the thermal conditioning components are secured to a thermally conductive pad, mat or similar member 350. The thermally conductive mat 350 can be formed from a plurality of thermally conductive fibers or filaments including metallic fibers (e.g., copper fibers, steel fibers, aluminum fibers, fibers of other metals or alloys, etc.), nonmetallic fibers (e.g., carbon fibers), and/or any other fiber comprising at least partially thermally conductive materials. In some embodiments, the fibers are arranged or otherwise organized into a bundled or meshed structure, similar to that present in steel wool or copper wool. In some embodiments, the thermoelectric device or other thermal conditioning device included in a skin conditioning system can be secured directly or indirectly to at least a portion of the a thermally conductive mat, pad or other member (e.g., using soldering, welding, adhesives, screws, rivets, tabs, clips, other mechanical fasteners and/or any other attachments devices or methods). In other embodiments, the thermal conditioning devices (e.g., thermoelectric devices, other cooling or heating devices, etc.) are indirectly secured to the thermally conductive pad using one or more intermediate members that are thermally conductive.

In some embodiments, the thermally conductive pad or mat 350 is relatively thin, having a thickness of between about 1 mm to about 30 mm, between about 3 mm to about 20 mm, between about 5 mm to about 10 mm, and any other value as desired or required. In some embodiments, the width of the fibers included in a thermally conductive pad or mat can be between about 0.5 mm to about 0.001 mm, between about 0.4 mm to about 0.005 mm, between about 0.3 mm to about 0.01 mm, between about 0.2 mm to about 0.05 mm, and any other value as desired or required. A thermally conductive fibrous structure can allow, in certain arrangements, for desirable thermal conduction through the mat while also providing breathability through the fibrous structure. Accordingly, perspiration from the subject being treated with the thermally conditioning system can be allowed to more easily evaporate through the structure. This can help eliminate or reduce the likelihood of having perspiration or other moisture trapped on the subject's skin, thereby increasing user comfort, increasing the efficiency and/or efficacy of a particular thermal conditioning procedure and/or providing additional benefits and advantages. Alternatively or additionally, a porous, conductive fibrous structure can provide convective conditioning by allowing air to be forced through passages or pores within the structure. Moreover, a thermally conductive fibrous structure can provide a degree of resiliency to enhance contact with the targeted skin surface of the subject and to enhance conformity over uneven portions of the subject's skin surfaces.

In any of the embodiments described herein, one or more intermediate layers I positioned between a thermally conditioning assembly or device and the subject's skin or other target anatomical surface can include a structure of one or more materials that comprise fluid-like properties. For example, in some embodiments, such a thermally conductive slurry comprises one or more metals (e.g., copper) in a liquid suspension. Thus, such a structure or slurry can be configured to conform substantially to the user's skin surface (e.g., like an icepack). The resulting structure or slurry of material can include amorphous solids such as, for example, gels, ground solids (e.g., metallic powders), fluids (e.g., liquids or other mixtures), and/or any other materials, solely or in combination with one another and/or any other substance or material (e.g., fillers, additives, etc.), as desired or required. For example, in some embodiments, copper powder can be contained within a flexible pouch and used as an intermediate layer I. Accordingly, the copper powder can beneficially retain the advantageous heat conductivity characteristics of solid copper while being significantly more conformable than the solid. Other types of materials can be mixed in the “slurry” to create a structure having favorable thermal conductively properties, as desired or required.

In other embodiments, a flexible band or strap can be positioned or otherwise located between the thermal conditioning members (e.g., thermoelectric devices, other cooling or heating devices, etc.) and the subject's skin or other anatomical surface being treated. Such flexible bands can comprises one or more materials having favorable thermal conductivity properties, such as metals (e.g., copper), alloys, other synthetic or natural materials, etc. In some embodiments, such bands or straps are integrated into a single structure with the thermal conditioning elements and/or the rest of the thermal conditioning system so as to form a unitary design. However, in other embodiments, such bands or straps can be separate and distinct from the thermal conditioning devices and/or other components of the system. Accordingly, in some arrangements, the strap, band or other member that contacts the subject during a procedure can be disposable or removable (e.g., for washing, sterilization, re-use, recycling, etc.). Other intermediate layers or components disclosed herein, including but not limited to, bladders, conductive slurries or slurry layers, conductive fibrous pads or mats and/or the like, can be similarly configured to be integrated into the system design or to be separate from other system components, as desired or required.

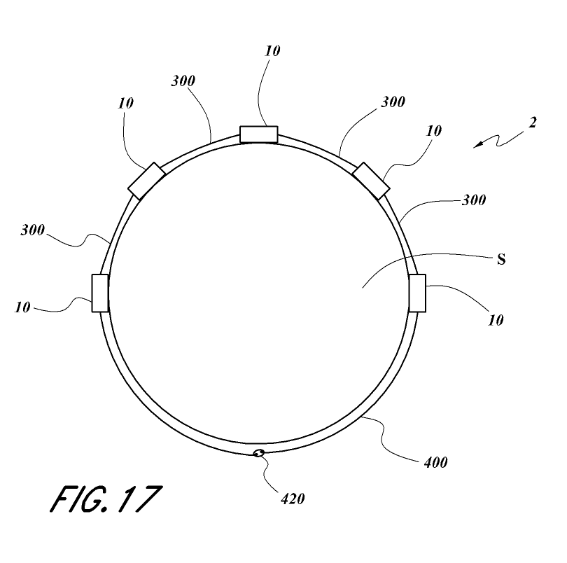

As illustrated in FIG. 17, a cooling or other thermal conditioning system 2 configured for placement around a subject's limb S, can include a plurality of heat transfer assemblies 10 (e.g., thermoelectric devices or other thermal conditioning devices, heat sinks and/or the like). As shown, adjacent assemblies 10 can be mechanically and/or electrically coupled to each other using one or more interconnecting members 300. In addition, a securement device 400 can be used to hold the system 2 in place during use. For example, the securement device or feature 400 can comprise a strap having a latch or other fastener 420 (e.g., Velcro, tab, other mechanical coupling, etc.). In some embodiments, the securement device or feature is at least partially flexible or extendable to facilitate securement of the system 2 to the subject S. As noted above, in the depicted arrangement or any other embodiment disclosed herein, the interconnecting members 300 and securement device 400 can comprise one or more thermally conductive materials, including, without limitation, metals (e.g., copper), alloys and/or the like. In some embodiments, the interconnecting members 300 and/or the device 400 comprise one or more resilient materials and/or structures, such that the assembly can generally conform to the subject's skin surface upon attachment.

As illustrated in FIGS. 18 and 19, in any of the embodiments as described herein, the thermal conditioning system 2 can also include one or more exterior layers 500 which entirely or partially cover the system 2, such as thermoelectric devices 100, heat sinks 20, intermediate layers I and/or any other component, as desired or required. In some embodiments, the exterior layers 500 are configured to at least partially thermally insulate certain or all components of the system 2 to enhance efficiency and/or efficacy of the device (e.g., provide improved heat transfer to and from the skin), reduce total energy usage and/or provide one or more other benefits and advantages. For example, by insulating the intermediate layers I, such as a conductive intermediate layer (e.g., conductive slurry, conductive copper band, conductive fibrous pad or mat, etc.) inefficiency caused by heat transfer from the intermediate layer I to the surrounding air can be reduced. In some embodiments, the exterior layer 500 can comprise one or more elastic or other resilient materials to facilitate retention of the system 2 on and/or conformity with the subject's body.

In some embodiments, such as those illustrated in FIGS. 18 and 19, an exterior layer 500 of the conditioning system can include one or more channels or passages 510 to facilitate removal of waste side heat, such as from heat sinks 20, from the thermoelectric device 100. For example, as disclosed herein, air or other fluid can be transferred across heat transfer devices or heat exchangers that are in thermal communication with one or more thermal conditioning devices (e.g., thermoelectric devices) to facilitate with the transfer of heat (e.g., waste heat) away from the thermal conditioning devices. Thus, in some embodiments, channels, either open or closed, can be used to direct, at least partially, the flow of air or other fluid originating from a blower, fan or other fluid transfer device to and through (and/or near) one or more of the thermal conditioning devices. As shown in the illustrated embodiments, the channels 510 can be longitudinally oriented from a first side to a second side of the system 2. In other embodiments, however, the channels can extend only partially along the system and/or can extend along a completely different orientation. In the illustrated arrangements, the channels comprise a generally rectangular shape. However, the shape, size and/or properties of the channels can vary. For example, the channels can include a different cross-sectional shape, such as, other polygonal, circular, oval, irregular and/or the like.

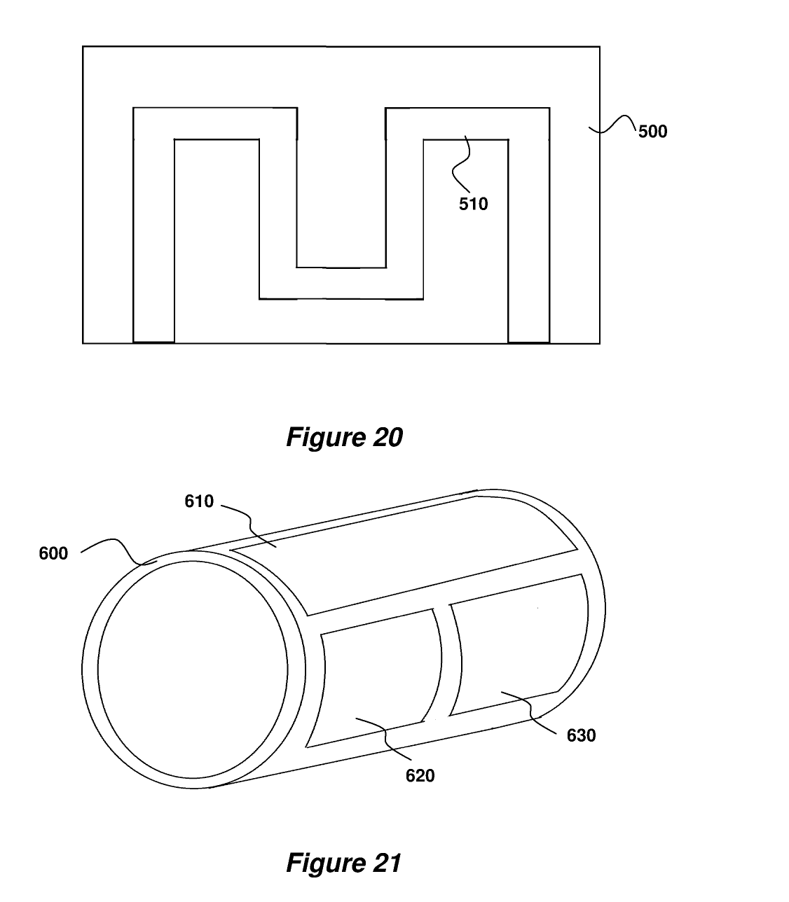

In some embodiments, the channels 510 can include other shapes (e.g., cross-sectional, shape of their layout relative to a thermal conditioning system, etc.) and/or can include curves or bends such as is shown in FIG. 20. The channels 510 can be “closed” such that they are exposed or open only at the terminal ends. This can advantageously prevent or reduce the likelihood of injury to the subject being treated or other user of the system, increase comfort and/or provide additional advantages by covering up the heat transfer members (e.g., fins, other heat sinks, etc.) 20 which may be uncomfortably, or otherwise undesirably, hot or cold. Moreover, closed channels 510 can be used to more effectively control the flow of fluids through the channel 510. For example, air or other fluid directed to a closed channel from a blower or other fluid transfer device will be directed from one end of the channel to the other end without escaping to the environment.

Alternatively, however, the channels 510 can be “open” such that a portion of the channel is exposed to the surrounding ambient environment. Accordingly, in some embodiments, an open channel can leave the heat transfer members (e.g., fins, other heat sinks, etc.) 20 at least partially exposed to the ambient environment, yet recessed in the exterior layer 500. Such configurations can be advantageous for enhancing heat transfer from the waste heat side to the surrounding environment, particularly when forced fluid flow across the heat transfer members 20 is not used. Moreover, the heat transfer members 20 can be positioned within an exposed recess of a channel and can be designed to remain below the outermost portions of the exterior layer 500. Thus, in such arrangements, the likelihood of contact between the subject (or other user, doctor, nurse, other practitioner or caregiver, etc.) and the heat transfer members (e.g., fins, other heat sinks, etc.), which may be undesirably hot or cold during use, can be reduced. Further, in some embodiments of an open channel configuration, the channel is only partially open. For example, in such an embodiment, the channel 510 can include openings or ventilation holes along outer peripheral surface.

In some embodiments, one or more fluid transfer devices 520, such as fans or pumps, can be included in the channel 510 to generate forced fluid flow through the channel 510. In other embodiments, however, such a fluid transfer device is separate from the channel, but placed in fluid communication with it (e.g., using a connecting conduit). As discussed herein, the use of a blower or other fluid transfer device can increase the rate of heat removal from the waste heat side of the thermoelectric device 100 or other thermal conditioning device by forcing fluid across the heat transfer members (e.g., heat sink) 20. In embodiments having a partially open channel structure, the fluid flow device 520 can receive ambient air from outside the exterior layer 500 through one or more apertures or other openings 530 proximate the fluid transfer device 520. Such apertures can be adjacent, for example, an intake portion of the fluid flow device 520. The fluid transfer device can be configured to deliver fluid flow in opposite directions through the channel 510, as shown, for example, by arrows 540, 545 in the embodiment of FIG. 18. In embodiments having a closed channel structure, the fluid transfer device 520 can receive fluid from a first opening to the opposite, second opening of the channel 510 as shown by arrows 550, 555 in FIG. 18.

In some embodiments, a closed channel 510 can form part of a liquid loop cooling system, wherein liquid is delivered through, past or near heat exchange member 20 to advantageously transfer heat therefrom. A closed channel 510 configured to receive liquid or other fluid can be a completely closed loop, such that, during operation, liquid remains contained within the channel 510. A fluid transfer device 520 can be used to circulate the liquid through the channel 510, past the heat exchange members 20 in order to selectively remove heat from (or transfer heat to) such members. In some embodiments, the liquid can be used to transfer heat from heat transfer members 20 located adjacent the thermoelectric devices 100 or other heat transfer devices to a radiator or separate heat transfer device located elsewhere on the system 2. In some embodiments, the channel 510 itself can serve as a heat sink and radiator device.

The system of channels 510, such as the liquid loop cooling system, can be included in an exterior layer 500 having a generally rigid structure. A rigid exterior layer 500 can advantageously be used as an immobilizing cast to stabilize and hold anatomical structures, such as bones and/or joints, to prevent or reduce the likelihood of further injury to those anatomical structures. In some embodiments, such exterior layers that define one or more channels through which fluid may pass (e.g., to transfer heat away from heat exchange members or other portions of a thermal conditioning device during use) can extend continuously or intermittently along the channels. In some embodiments, such exterior or shield layers or members can be removable (and/or replaceable) to provide access to the thermal conditioning devices, the channels and/or any other components positioned underneath or within the exterior or shield layers. In some embodiments, the exterior or shield layers or members are at least partially breathable, flexible, heat/fire resistant and/or the like, as desired or required.

As illustrated in FIG. 21, in any of the embodiments disclosed herein, one or more of the intermediate layers of the system 2 can comprise an expandable bladder or other expandable member 600. In some embodiments, one or more of the exterior layers 500 of the system can comprise an expandable bladder or other expandable member, either in lieu of or in addition to including an expandable bladder as an intermediate layer between the subject and a thermal conditioning assembly.

In some embodiments, the expandable bladder 600 can be used to selectively alter the pressure applied by the system 2 to the body part to which it is attached or along which it is adjacent (e.g., arm, hand, leg, foot, neck, other limb, etc.) and/or to provide a seal (e.g., partial, complete) between the system 2 and the body part. Altering the pressure around a body part can provide an additional therapeutic benefit to the user in certain situations. Moreover, in conjunction with the thermal conditioning treatment as herein described, pressurization can provide additional benefits and advantages. For example, altering such pressure can advantageously vary the thermal transfer rate by, among other things, varying contact resistance, compressing tissue, varying blood flow in the area of compression and/or the like. The seal created by the bladder 600 can be used to pressurize or create a vacuum within a cavity formed around a treatment area (e.g., cavity C, FIG. 8b).

As illustrated in FIG. 21, an expandable bladder 600 can include multiple chambers, such as chambers 610, 620, 630, to provide selective pressurization along certain localized zones. In some embodiments, such pressurization can be sequenced to provide additional therapeutic benefit to the user. For example, a first chamber 610 can be pressurized (or pressurized at a higher pressure level) while other chambers 620, 630 are depressurized (or pressurized at a lower pressure level). Subsequently, chamber 620 can be pressurized (or pressurized at a higher pressure level) while chambers 610 and 630 are depressurized (or pressurized at a lower pressure level). Further, as a subsequent step in a pressurization sequence, chamber 630 can be pressurized (or pressurized at a higher pressure level) while chambers 610, 620 are depressurized (or pressurized at a lower pressure level). In some embodiments, the cycle can be repeated for a desired time period. In other embodiments, the pressurization level along one or more portions or regions of the bladder or other expandable bladder can be different than discussed above. For example, a treatment procedure can include a customized pressurization scheme, as required or desired by a specific application, protocol or use. In other embodiments, a pressurization sequence can be regulated, at least in part, by feedback received from one or more sensors, timers (e.g., based on elapsed time) and/or the like. For example, the pressurization scheme associated with an expandable bladder or other expandable member can depend, at least in part, on feedback received from one or more of the following: temperature sensor, humidity sensor, perspiration or other condensation sensor, ion or chemical sensor, pressure sensor, heart rate monitor, blood pressure monitor, patient movement sensor or detector (e.g., accelerometer) core temperature measurements or feedback, skin conductance, oxygenation level (e.g., oxygen sensor), ambient conditions (e.g., ambient temperature, ambient relative humidity level, etc.), time of day and/or the like.

In some embodiments, one or more fluid transfer devices (e.g., blower, fans, pumps, etc.), valves (e.g., automatic or manual) and/or other hydraulic devices or members can be placed in fluid communication with the various fluid pockets or chambers included in a bladder or other expandable member in order to accomplish the selective pressurization (e.g., filling of fluid) and depressurization (e.g., evacuation of fluid). Such components can be operated using an automated control scheme in order to maintain the desired pressurization level and sequence. However, in other embodiments, one or more components of the pressurization system can be manually controlled, as desired or required.

In some embodiments, as noted herein, the use of bladders (e.g., balloons) and/or other expandable members in a thermal conditioning system can allow the application of pressure to the subject's skin surface or other anatomical region during the execution of a thermal conditioning procedure. The use of pressure can, in certain arrangements, impact the transfer of heat to and/or from the skin of the subject. For example, such pressure can vary contact resistance, can cause compression of skin and/or other tissues of the subject (e.g., which may affect one or more physiological responses of the subject), can vary blood flow in areas adjacent pressure application. In some embodiments, a bladder or other expandable member can be positioned completely or intermittently around one or more thermal conditioning devices (e.g., thermoelectric devices). Such bladders or other members can be incorporated into a thermal conditioning system (e.g., as a unitary or monolithic structure). Alternatively, however, a bladder can be a separate device or component that can be used in conjunction with a thermal conditioning system. In some embodiments, the bladder can comprise an inflatable cuff, similar to, for example, a blood pressure measurement cuff. The bladders 600 can include one or more attachment devices or methods for securing to itself, the subject, a portion of the thermal conditioning system (e.g., in embodiments where the bladder is separate and distinct from the system) and/or the like, including, but not limited to, straps, hook and loop fasteners, buttons, snaps adhesives and/or the like.

In any of the embodiments disclosed herein, the heat sink (e.g., heat sinks 20, 20′) can be extruded as one, two or more portions. In heat sink arrangements having two or more portions, such separate portions (e.g., fins, pins, base, etc.) can be attached to each other using welds, adhesives, other bonding agents, mechanical connections or fasteners, etc. In some embodiments, for example, the heat sinks comprise an extruded aluminum shape.

In any of the embodiments disclosed herein, a spacing between thermal assemblies 10 (e.g., thermoelectric devices) can be based on the body's thermo-regulatory response, which can naturally spread the effect of cooling or heating to an area larger that the physical footprint of the individual assemblies 10.

In any of the embodiments disclosed herein, the various assemblies 10 (e.g., thermoelectric devices) of a system can be arranged on a pad or other base member, with spacing of the assemblies 10 controlled, at least in part, by the structure of the pad or other base member. The spacing of the assemblies can be maintained as a result of the stiffness of the pad structure.

For any of the embodiments disclosed herein, the various thermoelectric devices and/or other thermal conditioning devices 100 of the system 2 can be configured in a series and/or parallel arrangement, as desired or required. Accordingly, the cooling system 2 can be configured to continue to function even if one or more individual thermoelectric devices or other thermal conditioning devices 100 were to fail to operate as desired. In some embodiments, a series and/or parallel arrangement of thermoelectric devices and/or other thermal conditioning devices 100 can be configured so that power is relatively uniform to the thermoelectric devices 100 that are in use.

In some embodiments, a system can be configured to enable a user to select which (and/or to what extent) the various thermal transfer assemblies (e.g., thermoelectric devices or other thermal conditioning devices) are activated at any particular time. For example, in some embodiments, the specific thermoelectric devices can be selected using a control module and/or locally at each device (e.g., via a switch or other controller).

For embodiments where temperature regulation or monitoring is desired (e.g., to prevent low or high operating or treatment temperatures), one or more thermal fuses can be incorporated into the system, either in addition to or in lieu of other temperature detection and/or control devices (e.g., thermistors, temperature sensors, etc.). Accordingly, each thermoelectric device and/or other thermal conditioning device 100 can have its power automatically (e.g., fully or partially), based on, for example, a skin temperature, a temperature of the thermal conditioning device or assembly, a temperature of the base layer of the system and/or the like.

In any of the embodiments disclosed herein, a thermal conditioning system can be configured for alternating heating and cooling of the adjacent skin of a subject. Such alternating heating and cooling of the subject's skin can offer one or more therapeutic or other benefits or advantages (e.g., promoting thermal transfer, promoting a desired therapeutic response, maintaining the body temperature within a particular range, etc.). In some embodiments, a heat sink (e.g., fins, pins, other heat transfer members, etc.) in thermal communication with a thermoelectric device or other thermal conditioning device 100 can be sized, shaped and/or otherwise configured to take advantage of the thermal mass of the heat sink and the transient condition of operation. For example, when the thermoelectric device is being operated in cooling mode, the heat sink temperature can increase with time. When, however, the thermoelectric device is switched to heating mode, the thermoelectric device can cool the heat sink. Accordingly, the temperature of the thermoelectric device can swing up and down when the device is switched between heating and cooling modes. Thus, in some embodiments, the size, thermal mass and/or other features of the heat sink can be advantageously selected based on the expected switching between heating and cooling mode of the system.

In some embodiments, cycling between a heating cycle and a cooling cycle (and/or modulation between different heating and/or cooling levels during the duration of a thermal conditioning procedure performed on a subject) can be based, at least in part, on a control scheme that incorporates one or more inputs, desired operational parameters and/or results, and/or any other considerations. For example, in some embodiments, such cycling is based, at least in part, on one or more temperature measurements (e.g., the temperature difference between the main side of the thermal conditioning device and the temperature of skin in contact with, or proximate, the thermal conditioning device, the temperature difference between the main and waste sides of the thermal conditioning device, the thermal transfer rate, maximum and/or minimum temperatures of the skin/system interface, of the thermal conditioning device or a component thereof and/or the like, etc.), other types of measurements (e.g., detection of perspiration or other condensation along or near the subject's skin), ambient conditions (e.g., ambient temperature, ambient relative humidity, time of day, etc.), pressure sending (e.g., created by a bladder or other expandable member, either incorporated into the skin cooling system or separate from the system), one or more physiological parameters of the subject and/or any other parameters as desired or required.

For example, in some embodiments, the cycling and/or modulation of cooling and/or heating provided to a subject is based on one or more physiological parameters of the subject, including, but not limited to, subject movement (e.g., as detected by one or more pressure sensors, accelerometers, other motion detectors, etc.), heart rate or pulse, blood pressure and/or other blood flow measurements or characteristics, variations in heart rate, blood pressure or other parameters, temperature measurements (e.g., core temperature, skin temperature, etc.), presence/amount of perspiration or other condensation along the skin and/or other anatomical area of a subject, skin conductance, profusion, oxygenation of blood (e.g., using an oxygen sensor) and/or the like.

The cycling (e.g., switching between cooling and heating, changing the level of cooling and/or heating, etc.) of the thermal conditioning system (and/or portions thereof, e.g., zones) can be controlled automatically or manually. For example, according to some embodiments, the cycling (e.g., modulation) of the various thermal conditioning devices can be based on a predetermined control scheme or routine. In some arrangements, a user can choose one of several different automated control schemes, as required or desired. For example, control schemes can be based, at least in part, on one or more of the following: type of condition that the subject is facing and that is desired to be treated (e.g., hyperthermia, hypothermia, etc.), the maintenance of a particular temperature effect (e.g., generally in terms of the level and degree of cooling or heating, specifically in terms of achieving a particular temperature along the subject's skin, etc.), the physiological parameters of a subject (as discussed herein) and/or other characteristics of the subject (e.g., age, gender, health condition, etc.) and/or the like. Such schemes can include one or more closed loop feedback control considerations, such as feedback based on a measurement or reading. Alternatively, such schemes can be predetermined routines that do not take into consideration any inputs.

In some embodiments, the thermal conditioning system can be configured to alternate between heating and cooling cycles (and/or between higher and lower degrees of cooling and/or heating). In such arrangements, one of the cycles (e.g., heating or cooling, higher or lower level of heating or cooling, etc.) can have a duration which exceeds the duration of another (e.g., opposite) cycle. In some embodiments, the cycling occurs between more than two levels of heating and/or cooling, such as, for example, 3, 4, 5, 6, 7 different heating/cooling settings or levels, more than 7 settings or levels, etc. By way of example, the cooling cycles can have a longer duration than the heating cycles such that the user is subject to a “net” cooling effect or the heating cycles can have a longer duration than the cooling cycles such that the user is subject to a “net” heating effect.

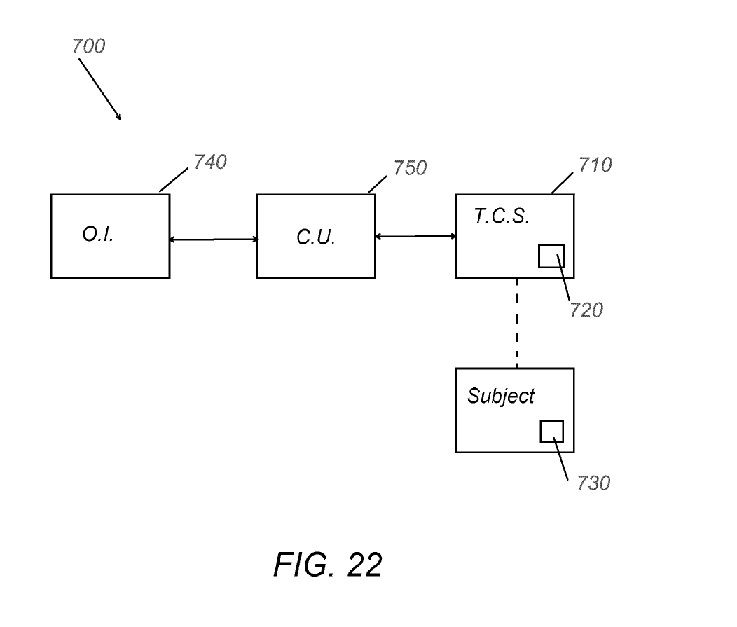

One embodiment of a thermal conditioning control system 700 is schematically illustrated in FIG. 22. The control system 700 includes a thermal conditioning system 710, system sensors 720, subject sensors 730, an operator interface 740, and a control unit 750. The thermal conditioning system 710 is configured to selectively heat and/or cool a subject and can be any one of the thermal conditioning system described herein, such as the thermal conditioning systems 20. As discussed in greater detail herein, the control system 700 can be configured to operate based on a closed-loop scheme using input from one or more feedback mechanisms (e.g., sensors 720, 730, operator interface 740, and/or other data, etc.).

The system sensors 720 sense or measure one or more operating conditions or other parameters of the thermal conditioning system 710 and output signals indicative of the sensed or measured parameter. In various embodiments, the system sensors 720 can include one or more of the sensors 200.

The subject sensors 730 sense or measure one or more physical conditions, physiological responses, or other parameters of the subject and output signals indicative of the sensed or measured parameters. In various embodiments, the subject sensors 730 can include one or more of the sensors 200, for example temperature sensors, heat flux sensors, humidity sensors, condensation/moisture sensors, pressure sensors, motion sensors, heart rate sensors, blood-oxygen sensors, galvanic (skin conductance) sensors, and any other type of subject sensor, as desired or required.

The operator interface 740 is configured to receive various control inputs, for example inputs by the subject or the subject's caregiver (e.g. doctor, nurse, or other treatment administrator), and communicate with the control unit 750.

The control unit 750 is configured to communicate with and control operation of the various components of the thermal conditioning system 710 and thereby controls a temperature of the subject and/or administers a thermal treatment to the subject. The control unit 750 controls operation based on inputs received from the system sensors 720, the subject sensors 730, and the operator interface 740. The control unit 750 can include a processing unit that if configured, based on the inputs it receives, to modify operation of the system based on a particular operational mode or scheme that has been selected based on the inputs and/or via the operator interface 740. In some embodiments, for example, the control unit 750 can modify an operational parameter of one or more components of the thermal conditioning system 710, including, but not limited to, the thermoelectric devices and/or other thermal conditioning devices or assemblies, fluid transfer devices (e.g., for passing air or liquid adjacent fins or other heat transfer members, for varying the pressure created by a bladder or other expandable member, etc.). Such control unit features can enhance safety for the subject and/or any user of a thermal conditioning system (e.g., preventing or reducing the likelihood of excessive cooling or heating of skin or other tissues), improve the comfort to the subject, provide for better therapeutic results for the subject, provide more predictable and repeatable results and/or one or more other benefits or advantages.

As discussed in greater detail herein, the control system 700 can be configured to operate based on a closed-loop scheme using input from one or more feedback mechanisms (e.g., sensors 720, 730, operator interface 740, and/or other data, etc.). As described above, the skin conditioning system can include one or more sensors such as temperature sensors, heat flux sensors, humidity sensors, condensation/moisture sensors, pressure sensors, motion sensors, heart rate sensors, blood-oxygen sensors, galvanic (skin conductance) sensors, and any other type of sensor, as desired or required. The control unit can include a processing unit that if configured, based on the inputs it receives, to modify operation of the system based on a particular operational mode or scheme that has been selected. In some embodiments, for example, the control unit can modify an operational parameter of one or more components of the system, including, but not limited to, the thermoelectric devices and/or other thermal conditioning devices or assemblies, fluid transfer devices (e.g., for passing air or liquid adjacent fins or other heat transfer members, for varying the pressure created by a bladder or other expandable member, etc.). Such control unit features can enhance safety for the subject and/or any user of a thermal conditioning system (e.g., preventing or reducing the likelihood of excessive cooling or heating of skin or other tissues), improve the comfort to the subject, provide for better therapeutic results for the subject, provide more predictable and repeatable results and/or one or more other benefits or advantages.

According to some embodiments, the control unit 750 can monitor one or more temperatures, power, or operational parameters of the thermoelectric devices or other thermal conditioning devices. For example, a sensor can be positioned to detect the temperature along the main side and/or waste side of a thermoelectric device. In some embodiments, in the event that the main side temperature detected by a temperature sensor (e.g., a thermistor) exceeds a high temperature limit or falls below a low temperature limit, the control unit 750 can operate one or more of the thermoelectric devices at a reduced power state or shut off entirely (e.g., for the safety of the subject and/or user of the system, to protect one or more components of the system against overheating or overcooling, etc.). Likewise, the control unit 750 can control the main side temperature based on the measured or calculated waste side temperatures. In some arrangements, the main side temperature can also be controlled based on the measured or calculated difference between the main side temperature and the waste side temperature.

In other configurations, the control unit 750 can control the thermal conditioning system 710, at least in part, based on a measured or calculated heat flux. For example, in the event that the control unit 750 determines that a threshold heat flux has been provided over a particular time period, the control unit 750 can reduce or discontinue power to one or more thermal conditioning devices (e.g., thermoelectric devices) such that the devices are turned off or operate a lower power or duty cycle level. As with other arrangements disclosed herein, the use of heat flux can help ensure that the subject and/or the user of a thermal conditioning system is safeguarded, that the system itself is not damaged and/or the like. In some embodiments, the use of heat flux can be used in combination with one or more temperature readings (e.g., temperature of a thermal conditioning device or component thereof, temperature of the subject's skin or other targeted anatomical area being treated, etc.). Such a configuration can provide, in certain arrangements, a more accurate measurement and understanding of total heat being applied or removed from a subject's body.

As discussed in greater detail herein, one or more other parameters can also be considered by the control unit 750 in the regulation of the thermal conditioning system 710. For example, the thermal conditioning control system 700 can include a motion sensor (e.g., accelerometer) as one of the subject sensors 730. In such systems, if the thermal conditioning control system 700 detects little to no subject motion, which may indicate that the thermal conditioning system 710 has been removed by the subject or that the subject has fallen asleep or has otherwise become unconscious, the control unit 750 can operate the thermal conditioning devices (e.g., thermoelectric devices, other cooling or heating devices, etc.) at a lower level to reduce the amount of thermal conditioning or can discontinue operation of the thermal conditioning system 710. In this way, the control unit 750 can protect the subject against potentially harmful excessive thermal conditioning. In some embodiments, the control unit 750 can restore operation of one or more devices of the thermal conditioning system 710 a previous operating level or to a higher level once the motion detector detects some subject movement or other activity that suggests the subject is awake and alert.

In some embodiments, the thermal conditioning control system 700 can comprise (and/or be operatively coupled to) a blood-oxygen sensor to detect the saturation of oxygen in a subject's blood, a heart rate monitor to detect the subject's heart rate, a blood pressure measurement device (e.g., an automated blood pressure measurement cuff or other device), a core body temperature sensor, perspiration or condensation sensor, skin conductance sensor, blood flow sensor, perfusion sensor and/or the like. Such physiological monitors can be useful indicators of a subject's status and can be incorporated into a control routine employed by the control unit 750, for example for turning one or more thermal conditioning devices on or off, modulating the operation of one or more conditioning devices between different operating levels, and/or the like as may be desired or required. The use of such feedback and other data regarding the subject, either alone or together with other inputs (e.g., temperature of the system and/or its components, ambient conditions, etc.), can be used by the control unit 750 to adjust or otherwise regulate the operation of the thermal conditioning system 710. By way of example, in the event that the subject's oxygen saturation, blood pressure, core temperature, heart rate and/or any other physiological property is above or below certain levels, the control unit 750 can adjust power to one or more of the thermoelectric devices or other thermal conditioning to a particular control scheme or routine.

In some embodiments, the thermal conditioning control system 700 can achieve a desired physiological response during different periods by controlling different combinations of subject parameters and thermal conditioning system parameters during these periods. For example, the thermal conditioning system 700 can achieve a desired change in body core temperature using different combinations of pressure and temperature applied by the thermal conditioning system 710 to the subject. More particularly, the thermal conditioning system 700 can vary the pressure to adjust blood flow rate, and can vary the temperature to adjust a temperature of the blood in the treatment area. In this and similar ways, the thermal control system 700 can operate at various different operating points for achieving a desired physiological response depending on other subject parameters.

In any of the embodiments disclosed herein, the various components of a skin conditioning system can communicate with one another using one or more wired or wireless methods. For example, in some embodiments, the thermal conditioning devices (e.g., thermoelectric devices) can be operatively coupled to a control unit, a user input device (e.g., remote control), a separate computing device (e.g., a personal computer, a tablet, a smart phone, etc.) using a hardwired connection and/or wirelessly (e.g., Bluetooth, Wi-Fi, LTE, WirelessHD and WiGig, other wireless protocol, etc.), as desired or required. Thus, is some embodiments, the system comprises or works together with one or more a wireless communication systems to wirelessly communicate with other device, components and/or the like, such as a control unit. In some embodiments, a control unit can include controls for altering operation of the system. Accordingly, the control unit can include buttons or other input features which allow a subject or another user to control one or more parameters of the system. For example, the control unit can allow a user to modify parameters such as temperature, pressure, cycling and/or any other parameter. The control unit can allow a user to select from a number of preprogrammed operational schemes (e.g., based on one or more inputs), to create a custom operational scheme (e.g., based on one or more inputs), to modify certain upper/lower limits (e.g., maximum or minimum temperatures, pressures, heart rate, core temperature and/or any other parameter described herein. In some embodiments, the control unit can include indicators such as lights, a display screen, audible sounds (e.g., alarms), or any other indicator, which provide information regarding the status of the system and/or the user of the device. Accordingly, this can allow the subject or a user or another party (e.g., doctor, nurse, other caregiver or practitioner, etc.) to monitor the subject and/or system and/or modify or otherwise control the execution of a particular cooling and/or heating procedure. In some embodiments, parts of the control unit, such as the processor, can be included in the control unit which can then wirelessly relay control signals to the system.

To assist in the description of the disclosed embodiments, words such as upward, upper, bottom, downward, lower, rear, front, vertical, horizontal, upstream, downstream have been used above to describe different embodiments and/or the accompanying figures. It will be appreciated, however, that the different embodiments, whether illustrated or not, can be located and oriented in a variety of desired positions.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited. The methods disclosed herein include certain actions taken by a practitioner or other caregiver; however, they can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 mm” includes “10 mm” Terms or phrases preceded by a term such as “substantially” include the recited term or phrase. For example, “substantially parallel” includes “parallel.”

1-71. (canceled)

72. A thermal conditioning system for selectively cooling or heating a skin surface of a subject, the system comprising:
at least one thermal conditioning device comprising a first side and second side, the second side being generally opposite of the first side;
a heat sink positioned along the second side of the at least one thermal conditioning device;
at least one intermediate member or base, the at least one intermediate member or base being configured to support the at least one thermal conditioning device, wherein the at least one intermediate member or base is configured to at least partially contact the skin surface of the subject during use;
wherein the first side of the at least one thermal conditioning device is configured to be placed in contact with or in close proximity to a skin surface of the subject to selectively cool or heat the skin surface.

73. The system claim 72, wherein the heat sink comprises one or more heat transfer members.

74. The system of claim 72, wherein the at least one intermediate member or base comprises a layer of fabric or plastic that is at least partially flexible so as to generally conform to a shape of the subject's skin surface.

75. The system of claim 72, wherein the at least one intermediate member or base comprises a thermally conductive slurry, solution or suspension, wherein the thermally conductive slurry, solution or suspension comprises a metal or an alloy.

76. The system of claim 72, wherein the at least one intermediate member or base comprises a thermally conductive flexible band, wherein the thermally conductive flexible band comprises a metal or an alloy and the metal or alloy comprises at least one of copper, aluminum and steel.

77. The system of claim 72, wherein the at least one intermediate member or base comprises a thermally conductive fibrous pad, wherein the thermally conductive fibrous pad comprises a metal or an alloy that is at least partially breathable, the metal or alloy comprising at least one of copper, aluminum and steel.

78. The system of claim 72, further comprising at least one expandable member positioned at least partially around the at least one at least one thermal conditioning device, the at least one expandable member being configured to create a controllable pressure on the skin surface of the subject when the system is in use.

79. The system of claim 89, wherein the at least one expandable member comprises a bladder.

80. The system of claim 72, wherein the system is configured to receive at least one physiological input regarding the subject, the system being configured to modify an operational parameter of the at least one thermal conditioning device based on the at least one physiological input, wherein the at least one physiological input comprises at least one of the following related to the subject: heart rate, blood pressure, core temperature, skin conductance,

81. The system of claim 72, wherein the heat sink extends at least partially within a channel, the channel being configured to receive a fluid for heat exchange between the heat sink and the fluid, wherein the channel is formed, at least in part, by an exterior layer or member.

82. The system of claim 81, wherein the channel comprises an open channel.

83. The system of claim 81, wherein the channel comprises a closed channel.

84. The system of claim 81, wherein the channel is configured to receive a gas.

85. The system of claim 81, wherein the channel is configured to receive a liquid, and wherein heat transfer with the heat sink is accomplished using a liquid-loop heat exchange system.

86. The system of claim 72, further comprising:
a control unit configured to control first and second operational parameters of the thermal conditioning device; and
a sensor configured to measure a physiological parameter of the subject an generate a sensor output indicative of a value of the physiological parameter,
wherein the control unit adjusts the physiological parameter of the subject from a first physiological value to a second physiological value different than the first value by adjusting the first operational parameter of the thermal conditioning device from a first control value to a second control value, and the control unit adjusts the second operational parameter of the thermal conditioning device based on the sensor output and at least one of the first control value and the second control value.

87. A method of selectively cooling skin of a subject, the method comprising:
placing a thermal conditioning system adjacent a skin surface of a subject, wherein the thermal conditioning system comprises at least one thermal conditioning device having a first side and second side, wherein the second side being generally opposite of the first side;
wherein the thermal conditioning system further comprises a heat sink positioned along the second side of the at least one thermal conditioning device, wherein the first side of the at least one thermal conditioning device is configured to be placed in contact with or in close proximity to a skin surface of the subject to selectively cool or heat the skin surface;
activating the at least one thermal conditioning device so at to selectively heat or cool the subject's skin; and
deactivating the at least one thermal conditioning device after a time period.

88. The method of claim 87, further comprising detecting a temperature of the at least one thermal conditioning device and/or the subject's skin using at least one sensor.

89. The method of claim 88, wherein the system is configured to deactivate when a threshold temperature is detected by the at least one sensor.

90. The method of claim 87, wherein the thermal conditioning system comprises a plurality of thermal conditioning devices, the thermal conditioning devices being arranged in at least two zones, wherein each of the at least two zones can be separately controlled and operated during use.

91. A method of selectively cooling skin of a subject, the method comprising:
placing a thermal conditioning system adjacent a skin surface of a subject, wherein the thermal conditioning system comprises at least one thermal conditioning device having a first side and second side, wherein the second side being generally opposite of the first side;
wherein a pressure exerted by the thermal conditioning system on the skin surface can be selectively modified to alter at least one physiological response;
wherein based on the pressure exerted on the skin surface, a particular output level of thermal conditioning created by the thermal conditioning system can create varying degrees of thermal conditioning to the subject.

92. The method of claim 91, wherein the pressure is exerted on the skin surface by a bladder.
